(19) United States
(12) Patent Application Publication
(10) Pub. No.: US 2024/0263141 A1
(43) Pub. Date: Aug. 8, 2024

(54) Tissue Extracts and Related Methods

(71) Applicant: ALLOSOURCE, Centennial, CO (US)

(72) Inventors: William Maslanik, Centennial, CO (US); Reginald Stilwell, Centennial, CO (US); Jeffrey Brown, Centennial, CO (US); Michael Floren, Centennial, CO (US)

(73) Assignee: ALLOSOURCE, Centennial, CO (US)

(21) Appl. No.: 18/567,470

(22) PCT Filed: Jun. 8, 2022
(86) PCT No.: PCT/US2022/072805

§ 371 Date: Dec. 6, 2023

Related U.S. Application Data
(60) Provisional application No. 63/208,766, filed on Jun. 9, 2021.

(51) Int. Cl. C12N 5/0775; A61K 35/28; C12M 1/00; C12M 1/42

(52) U.S. Cl. CPC C12N 5/0662; A61K 35/28; C12M 35/04; C12M 47/04; C12N 2501/10; C12N 2510/04

(57) ABSTRACT

Provided are enriched tissue extracts and methods of making and using such extracts. The enriched tissue extracts contain biologically active components. Some extracts produced by the methods contain extracellular vesicles. The methods can involve administering a physical stress on a biological sample comprising live cells or otherwise stimulating the live cells of the biological sample. Also contemplated are methods in which the biological sample does not contain live cells. The biological sample is incubated in an extraction solution for a period of time sufficient for extraction of biologically active components from the biological sample. Methods of using the extracts, or extracellular vesicles isolated therefrom, therapeutically and for in vitro purposes are also provided.

### CROSS-REFERENCE TO RELATED APPLICATIONS

This present application claims benefit of priority to U.S. Provisional Patent Application 63/208,766 filed on Jun. 9, 2021. The foregoing application is hereby incorporated by reference as if fully set forth herein in its entirety for all purposes.

### BACKGROUND

Embodiments of the present disclosure are directed in general to the field of tissue healing and cell growth, and in particular to methods for producing enriched tissue extracts for healing injured tissue and influencing cell growth.

Growth factors have been investigated for stimulation of healing and for research for many years. Growth factors have been applied in isolation or in combination with a small number of factors. Growth factors, along with other biologically active components, are released by cells in response to injury or disease (e.g., stress). The application of growth factors for stimulating healing of injured tissue often fails to trigger the full cascade of natural healing within the tissue for a variety of reasons. One such reason is that growth factors represent only a single segment of the biologically active components required to stimulate cellular healing. Moreover, growth factors only activate one form of tissue healing. Accordingly, there is still a need for improved therapeutics. Embodiments of the present disclosure provide solutions to at least some of these outstanding needs.

### BRIEF SUMMARY

In one aspect, provided are methods of producing an enriched tissue extract. The methods may include providing a biological sample; incubating the biological sample in an extraction solution for a period of time sufficient for biologically active components to be extracted from the biological sample thereby forming an enriched tissue extract; and separating the enriched tissue extract from the processed biological sample. In some embodiments, the biological sample may include live cells. For example, the biological sample may include isolated cells, primary cells, immortalized cells, and/or mesenchymal stem cells. In some cases, the isolated cells may be immortalized mesenchymal stem cells that are genetically modified to express at least one recombinant growth factor that is not normally expressed by mesenchymal stem cells or to overexpress a growth factor that is normally expressed by mesenchymal stem cells.

In some embodiments, the biological sample may include tissue or isolated cells from one or more of cancellous bone, cortical bone, cortical and cancellous bone, periosteum, ligament, tendon, muscle, placenta, amnion, or umbilical tissue. In some embodiments, the biological sample may include a single type of tissue and is substantially free of other types of tissue, while in other embodiments, the biological sample can include a portion of tissue or a plurality of tissue pieces. The biological sample may be from a deceased donor subject and/or a living donor subject. In some embodiments, the biological sample is from a human donor subject.

In embodiments, the methods described herein may include a step of administering a physical stress to the biological sample prior incubation. The physical stress may include one or more of a mechanical stress, a chemical stress, or a temperature stress. In exemplary embodiments, the step of administering a physical stress to the biological sample may induce a stress response in the live cells. In some cases, the step of administering a physical stress to the biological sample may result in minimal cell death, while in other cases, the step of administering a physical stress to the biological sample kills all or substantially all of the cells in the biological sample.

In some embodiments, administering a physical stress to the biological sample may include at least one of exposing the biological sample to sinusoidal tension, exposing the biological sample to compression or pressure, homogenizing the biological sample, exposing the biological sample to mechanical impacts, or exposing the biological sample to resonant acoustic energy. In other embodiments, administering a physical stress to the biological sample may include exposing the biological sample to hypothermic temperatures, hyperthermic temperatures, acidic conditions, osmotic stress, non-physiological pH, or non-physiological oxygen levels. In still other embodiments, administering a physical stress to the biological sample may include homogenizing, freezing and thawing the biological sample, cryofracturing the biological sample, or heating the biological sample above 45° C., and wherein administering the physical stress results in death of all or substantially all of the cells present in the biological sample. In further embodiments, the biological sample may be bone tissue and administering a physical stress to the biological sample may include demineralizing the bone tissue. In some cases, the biological sample may be exposed to one or more growth factors prior to incubation.

In some embodiments, the extraction solution that the biological sample is incubated in may include a buffer solution or a cell culture medium. In some cases, the extraction solution may include a salt, a serum, a detergent, and/or a protease inhibitor. In some embodiments, the biological sample is incubated in the extraction solution for a period of 5 minutes to 24 hours at 2° C.-42° C. Incubating the biological sample in the extraction solution may include agitating the biological sample in an extraction solution.

In some embodiments, separating the enriched tissue extract from the processed biological sample may include separating the enriched tissue extract from the biological sample using at least one of centrifugation or filtration. In some embodiments, the enriched tissue extract may include one or more of micro-vesicles, exosomes, growth factor proteins, nucleic acids, extracellular matrix proteins, or signaling molecules. Exemplary signaling molecules may include one or more of amino acids, hormones, neurotransmitters, cyclic AMP, or steroids.

In some embodiments, the methods described herein may further include adding to the enriched tissue extract at least one of a recombinant growth factor protein, a protease inhibitor, or a serum. In some embodiments, the methods described herein may further include concentrating the enriched tissue extract by one or more of precipitation, cellulose membrane concentration, dialysis, or chromatography. In some embodiments, the methods described herein may further include freezing the enriched tissue extract at −20° C. In such cases, the enriched tissue extract may be combined with a cryopreservation medium prior to freezing.

In another aspect, enriched tissue extracts produced by the methods described herein are provided. In another aspect, provided are methods of producing isolated extracellular vesicles, the methods including producing the enriched tissue extract, as discussed herein, and isolating extracellular vesicles from the enriched tissue extract. In some embodiments, the isolating extracellular vesicles from the enriched tissue extract step may include one or more of filtration, magnetic cell separation, or particle separation.

In another aspect, methods of treating a subject with a wound are provided herein. Such methods may include administering to the wound of the subject the enriched tissue extract described herein. In some embodiments, the method includes administering to the wound of the subject the isolated extracellular vesicles produced by the methods described herein.

In still other aspects, methods of in vitro culturing cells are provided. Such methods may include culturing cells in a cell culture medium including the enriched tissue extract described herein or the isolated extracellular vesicles produced by the methods described herein. In some embodiments, the cells that are cultured may include one or more of mesenchymal stem cells, tenocytes, fibroblasts, or osteocytes.

In another aspect, provided are methods of producing extracellular vesicles, the methods including in vitro culturing cells in a serum-free cell culture medium including the enriched tissue extract described herein or extracellular vesicles produced by the methods described herein. The methods may also include separating a supernatant including the serum-free cell culture medium and extracellular vesicles from the cells; and isolating the extracellular vesicles from the supernatant by membrane filtration or pelleting by centrifugation. In further aspects, a product including cultured eukaryotic cells that have been cultured in the presence of the enriched tissue extract or the extracellular vesicles produced by the methods described herein may be provided.

The above described and many other features and attendant advantages of embodiments of the present disclosure will become apparent and further understood by reference to the following detailed description when considered in conjunction with the accompanying drawings.

### BRIEF DESCRIPTION OF THE DRAWINGS

These figures are intended to be illustrative, not limiting. Although the aspects of the disclosure are generally described in the context of these figures, it should be understood that it is not intended to limit the scope of the disclosure to these particular aspects.

FIGS. 1A-1B shows steps in methods of processing tissue according to aspects of the present disclosure.

FIG. 2 shows a graph of tenocyte expansion obtained using the exemplary method of Example 1, according to aspects of the present disclosure.

FIGS. 3A-3D depicts images of extracellular matrix obtained using the exemplary method of Example 2, according to aspects of the present disclosure.

FIGS. 4A-4D depicts microscopy images of bone marrow MSC cultured with bone marrow extract for 24 hours as described in Example 3, according to aspects of the present disclosure. FIG. 4A shows a negative control of 40% bone marrow extract added to formalin fixed cells. FIGS. 4B-4D show the results of live MSC cultured with 10%, 20%, and 40% extract, respectively.

FIGS. 5A-5D depicts microscopy images of bone marrow MSC cultured with bone marrow extract for 24 hours as described in Example 4, according to aspects of the present disclosure. FIG. 5A shows a negative control of 40% bone marrow extract added to formalin fixed cells. FIGS. 5B-5D show the results of live MSC cultured with 10%, 20%, and 40% extract, respectively.

### DETAILED DESCRIPTION

This disclosure provides methods and compositions in the field of medical therapies, and particularly, relates to enriched tissue extracts and methods of producing enriched tissue extracts. The enriched tissue extracts as provided herein include biologically active components that are secreted or expelled by cells in a tissue when stressed. The enriched tissue extracts include tissue specific components that can induce specific type of tissue healing, as well as influence cellular growth and differentiation in a controllable manner. The enriched tissue extracts made by the methods described herein may be useful in various industries including, amongst others, regenerative therapies and research.

When tissue is injured, in a diseased state, or the integrity of the cell structure is otherwise disrupted, growth factors and other signaling molecules are released from the tissue to stimulate healing of the injury. For example, when tissue is injured, mesenchymal stem cells within the tissue may produce cytokines and growth factors to decrease inflammation, enhance progenitor cell proliferation, improve tissue repair and decrease infection. Current therapies use single growth factors or small combinations of growth factors which do not accurately initiate the natural cascade of healing events. Conventional pharmaceutical and therapies are often unable to adequately induce healing of injured tissue or cells because of reduced vasculature, prominent necrosis, or lack of required receptors to mediate uptake of the therapeutic components. Thus, treating injured tissue using conventional methods may only provide limited pro-healing therapeutics to the injured tissue and fail to initiate the cascade of natural healing within the injured cell tissue.

The enriched tissue extracts as provided herein may provide a broad array of pro-healing signals (e.g., signaling factors, growth factors) to injured tissues to induce a cascade of natural healing. The enriched tissue extracts herein may have superior biological function and activity than that of a single growth factor or a simple growth factor mixture. These extracts may change the standard practices of cell culture in that they may replace typical culture media supplements, such as growth factors or serum. In some cases, the enriched tissue extracts may enable culture of therapeutic cells without the addition of animal serum or proteins.

Moreover, the enriched tissue extracts herein may more efficiently and more effectively ameliorate disease by promoting secretion of paracrine acting factors, increase tissue healing by reducing inflammation, reprogram immune cells, activate endogenous repair pathways, and/or suppress specific cell proliferation. In some aspects, the enriched tissue extracts of the present disclosure may be used as therapeutics to heal injured tissues because of micro-vesicles and exosomes within the extract itself. Micro-vesicles and exosomes carry as cargo mRNAs, microRNAs, and proteins, and are able to function as paracrine mediators by horizontal transfer of this cargo during tissue repair. The enriched tissue extracts may allow delivery to the injured tissue because the pro-healing signals within the extract may not require receptors to mediate uptake into the injured tissue and may not require an oxygenated blood supply to reach the injured tissue.

The enriched tissue extracts herein are not naturally present in native tissue in the body. When tissue in its native environment (i.e. in a body) is exposed to injury or disease, any biological factors secreted from the live cells of the tissue interact with fluid and other components in the immediate interstitial region between affected cells. Additionally, extracellular factors are recruited to the site of injury or disease and influence repair and/or healing of the native tissue. In contrast, the enriched tissue extracts that are provided herein are separate from native environment of the cell or the tissue itself and constitute a collection of components that do not exist in an isolated state in the body. The enriched tissue extracts provided herein are concentrated mixtures of potent components in a form that can be readily applied to cells and tissues in vivo and ex vivo as desired. As such, as described herein, the enriched tissue extracts may be used in vitro and ex vivo to facilitate growth of cells (primary, cultured, recombinant) or cell growth and/or healing in a tissue in a subject, such as a surgical site or wound. In some instances, the extracts contain only soluble fractions of components secreted by the cells or tissues used to produce them. In such instances, the enriched tissue extracts may not contain surfactants or detergents that would cause total cell lysis. In some instances, the extracts are made from cells or tissue that have been cryofractured and thus may contain components that are typically matrix bound but are released into a soluble fraction by the cryofracturing process.

The use of enriched tissue extracts as provided in this disclosure to stimulate healing is counterintuitive because numerous prohibitive elements, such as proteases and degradative compounds also present in the enriched extracts, would be expected to render the stimulatory effects inactive. This is especially true when the enriched tissue extracts are produced from tissue comprising few to no living cells. The enriched tissue extracts according to the present disclosure, however, surprisingly induce and increase healing of an injured tissue and promote cell growth.

### I. Methods of Producing Enriched Tissue Extracts

The methods provided in this disclosure may be used to produce enriched tissue extracts having different therapeutic effects and/or for different tissue types, depending on the type of biological, the extraction solution, and type of stimulation applied to the biological sample.

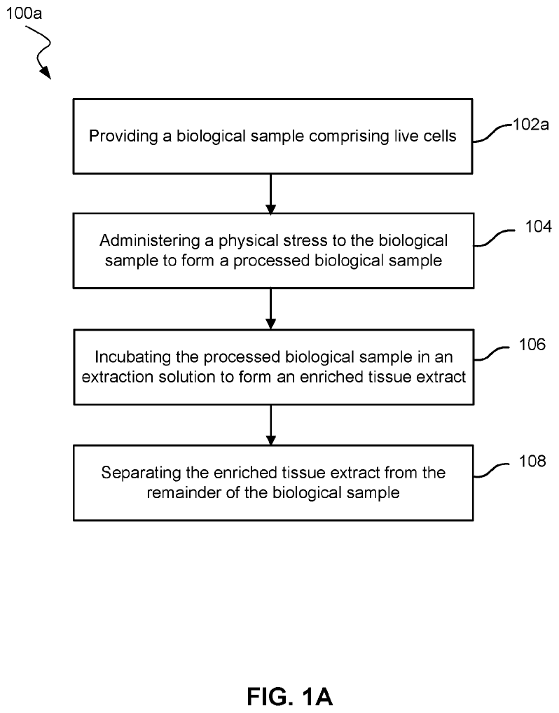

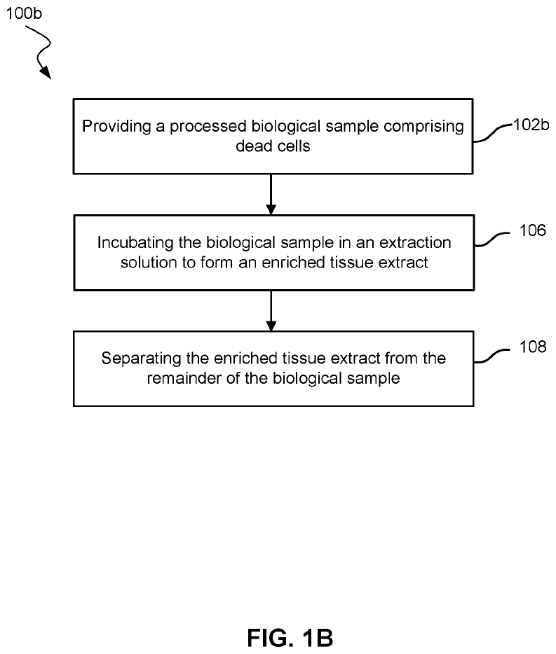

FIG. 1A and FIG. 1B show exemplary methods 100a and 100b for producing an enriched tissue extract according to aspects of the present disclosure. The method include a step of providing a biological sample for extraction. In some embodiments, as shown in FIG. 1A, the biological sample provided in step 102a includes live cells. In other embodiments, as shown in FIG. 1B, a processed biological sample is provided in step 102b that primarily includes lysed or otherwise dead cells (i.e. substantially all or all of the cells present are lysed or otherwise dead). For example, the processed biological sample may be a biological sample that is frozen and thawed in prior to step 102b, thereby such processing lysing at least a portion of the cells within the biological sample. Optionally, methods 100a and 100b may include a step of cleaning the biological sample.

The method shown in FIG. 1A includes optional step 104 of administering a physical stress on the biological sample (i.e. stimulating the biological sample) to produce a processed biological sample. In step 104, physical stress may include one or more of a mechanical stress, a chemical stress, or a hypothermic stress. In some embodiments, the physical stress may induce a stress response in the live cells of the biological sample. In other embodiments, the physical stress may disrupt the structural integrity of cells within the biological sample. In step 104, physical stress may be applied to the biological sample for a specified duration of time, depending on the physical stress applied. Step 104 may be repeated a plurality of times. Each application of physical stress to the biological sample may be considered one cycle. In some instances, when step 104 is repeated (such as when method 100 comprises multiple cycles), a different means of physical stress may be used for each cycle within step 104.

Following administering the physical stress at step 104 of method 100a shown in FIG. 1A or following step 102b of method 100b shown in FIG. 1B, the next step may be step 106. At step 106, the processed the biological sample produced by step 104 or provided in step 102b may be incubated in an extraction solution to form an enriched tissue extract. The biological sample may be incubated in the extraction solution for a period of time sufficient for biologically active components to be extracted from the processed biological sample of method 100a or the biological sample of method 100b. In some instances, in method 100a where the biological sample contains live cells, the live cells in the processed biological sample secrete biologically active components into the extraction solution. In some instances, at least some of the live cells in the processed biological sample are permeabilized such that biologically active components pass from inside the live cells and into the extraction solution. In other cases, in either method 100a or 100b, a disruption in the structural integrity of the cells within processed biological sample causes expulsion or secretion of the biologically active components from processed biological sample into the extraction solution. The biologically active components extracted from the processed biological sample may form an enriched tissue extract.

Methods 100a and 100b may include step 108 in which the enriched tissue extract is separated from the tissue and cells of the processed biological sample. Optionally, at least one of a serum, one protease inhibitor, a recombinant growth factor protein, and/or an antibiotic may be added to the enriched tissue extract, in some instances as part of the extraction solution (step 106) and/or in other instances after the separation step (i.e. via an additional step). In some instances, method 100 may also include a step of concentrating the enriched tissue extract. Concentrating the enriched tissue extract may include one or more of precipitation, cellulose membrane concentration, dialysis, or chromatography. After separation of the enriched tissue extract at step 108, the enriched tissue extract may be frozen, such as by known cryopreservation means, at a temperature of at −20° C.

It has been discovered that the enriched tissue extracts produced via the methods described herein can directly stimulate healing of a tissue in an injured or diseased state. In some aspects, the enriched tissue extract can stimulate cells in in vitro cultures to proliferate, secrete soluble factors, or stimulate stem cells to differentiate for use in clinical products or for in vitro cell culture purposes. Cell function and phenotype may be maintained during cell cultures using the enriched tissue extracts herein. Moreover, it has been discovered that the enriched tissue extracts can stimulate live cells to produce extracellular matrixes, expand at faster rates, and/or produce extracellular vesicles, including exosomes and secretomes. It is these properties of that allows the enriched tissue extracts, upon administration to a subject, to initiate the natural cascade of healing events that prior therapies are unable to achieve. Similarly, the enriched tissue extract provided may be used for in vitro cell culture purposes to maintain, expand, and/or study mammalian cells.

### A. Biological Sample

### 1. Tissue

The biological sample used according to the methods of this disclosure may be or include a tissue biological sample (i.e. a portion of tissue). A variety of types of tissue may be used. Tissue for use in the provided methods includes, but is not limited to, bone, tendon, skin, cartilage, osteochondral, fascia, muscle, nerves, vascular tissue, birth, and adipose tissue. For example, the biological sample may include one or more of cancellous bone, cortical bone, cortical and cancellous bone, periosteum tissue, ligament tissue, tendon tissue, muscle tissue, placental tissue, amnion tissue, or umbilical tissue. In some embodiments, the biological sample may include dermal tissue, neural tissue, thyroid tissue, osteochondral tissue, or organ tissue. For example, the biological sample may include heart, lung, liver, pancreatic, bladder, brain and/or spinal cord, or kidney tissue.

In some embodiments, the biological sample comprises live cells, that is that the cells in the biological sample are primarily alive (i.e. a substantial number of cells in the biological sample are alive). In such embodiments, the tissue biological sample may be provided before significant cellular death occurs to the live cells within the biological sample. For example, the tissue biological sample may be less than 72 hours, in some cases, less than 24 hours from the time that the biological sample is obtained from a donor subject.

In other embodiments, the biological sample comprises dead cells, that is that the cells in the biological sample are primarily dead (i.e. a substantial number of cells in the biological sample are dead). For example, some of the cells within the biological sample may be dead or all of the cells within the biological sample may be dead. In some instances, living cells within the tissue biological sample may be lysed before or during the processes disclosed herein. For example, the biological sample may be frozen and then thawed prior to being extracted in accordance with the described methods. In such examples, freezing the tissue biological sample may lyse a substantial portion or all of the cells within the tissue biological sample

The tissue biological sample may be obtained from a donor subject. The donor subject may be a human donor or a non-human animal. Non-human animals include, for example, non-human primates, rodents, canines, felines, equines, ovines, bovines, porcines, and the like. In some instances, the tissue biological sample may be obtained from a human donor, or may be derived from tissue obtained from a human donor. In some instances, the tissue biological sample may be obtained from a patient intended to receive the enriched tissue extract such that the enriched tissue extract is autologous to the patient. In some instances, the tissue biological sample may be obtained from a subject other than the patient intended to receive the enriched tissue extract, wherein the subject is the same species as the patient, such that the enriched tissue extract is allogenic to the patient. In some instances, the tissue biological sample may be obtained from a donor subject that is a different species than the patient intended to receive the enriched tissue extract, such that the enriched tissue extract is xenogenic to a patient. For example in some instances, the tissue biological sample may be obtained from a non-human animal for administration to a human patient.

The tissue biological sample may be obtained from one or more donors. In some embodiments, the tissue biological sample is obtained from a single donor. In some embodiments, the tissue biological sample is obtained from multiple donors (e.g., two or more donors). In some embodiments, the tissue biological sample is obtained from a living donor. In some embodiments, the donor is a deceased donor (i.e., a cadaveric donor). In some embodiments, the donor is a deceased human donor (i.e., a cadaveric human donor). Generally, when the tissue biological sample is obtained from a deceased donor for use in producing the enriched tissue extracts provided herein, it is recovered within 72 hours of asystole, or the ischemic time has been less than 72 hours. In other words, the donor has been deceased for no longer than 72 hours.

The tissue biological sample may be machined, cut, or processed into a shape before processing using the methods described herein. Such shapes include any of those discussed in this disclosure. In some instances, the tissue biological sample may be machined, cut, or processed into shapes such as, but not limited to, a cube, a strip, a sphere, a wedge, a disk, or an irregular shape. In some instances, the shape of the biological sample may facilitate processing of the biological sample according to the methods of the present disclosure. The tissue biological sample can include a plurality of tissue pieces that are similar in size or a plurality of tissue pieces of different sizes.

In some instances, the tissue biological sample may be a single portion of tissue. In other instances, the tissue biological sample may be a plurality of tissue pieces. For example, the tissue biological sample may have a weight of 0.5 grams to 5,000 grams (e.g., from 0.5 grams to 4,500 grams, from 1 grams to 4,500 grams, form 25 grams to 4,500 grams, from 100 grams to 4,500 grams, from 500 grams to 4,500 grams, from 1,000 grams to 4,500 grams, from 2,000 grams to 4,500 grams, from 3,000 grams to 4,500 grams, from 4,000 grams to 4,500 grams, from 0.5 grams to 4,000 grams, from 1 grams to 4,000 grams, from 25 grams to 4,000 grams, from 100 grams to 4,000 grams, from 500 grams to 4,000 grams, from 1,000 grams to 4,000 grams, from 2,000 grams to 4,000 grams, from 3,000 grams to 4,000 grams, from 0.5 grams to 3,000, from 1 gram to 3,000 grams, from 25 grams to 3,000 grams, from 100 grams to 3,000 grams, from 500 grams to 3,000 grams, from 1,000 grams to 3,000 grams, from 2,000 grams to 3,000 grams, from 0.5 grams to 2,000, from 1 gram to 2,000 grams, from 25 grams to 2,000 grams, from 100 grams to 2,000 grams, from 500 grams to 2,000 grams, from 1,000 grams to 2,000 grams, from 0.5 grams to 1,000 grams, from 1 gram to 1,000 grams, from 25 grams to 1,000 grams, from 100 grams to 1,000 grams, from 500 grams to 1,000 grams, from 0.5 grams to 500 grams, from 1 gram to 500 grams, from 25 grams to 500 grams, from 100 grams to 500 grams, from 0.5 grams to 250 grams, from 1 gram to 250 grams, from 25 grams to 250 grams, from 100 grams to 250 grams, 0.5 grams to 100 grams, from 1 gram to 100 grams, or from 25 grams to 100 grams).

In some instances, the tissue biological sample may be or include bone tissue. Bone is composed of organic and inorganic elements. By weight, bone is approximately 20% water. The weight of dry bone is made up of inorganic minerals such as calcium phosphate (e.g., about 65-70% of the weight) and an organic matrix of fibrous protein and collagen (e.g., about 30-35% of the weight).

The bone tissue may be cancellous bone or cortical bone. In some instances, the bone tissue is cancellous (trabecular) bone. Cancellous bone, also known as spongy bone, can be found at the end of long bones. Cancellous bone is typically less dense, softer, weaker, and less stiff than cortical bone. Cancellous bone may include bone growth factors. Cancellous bone has a trabecullar-like structure formed from an interconnected network of bone projections of variable thickness and length. The projections define voids in the bone. Cortical bone, also known as compact bone, can be found in the outer shell portion of various bones. Cortical bone is typically, dense, hard, strong, and stiff. Cortical bone may include bone growth factors. In some instances, the bone tissue may be cortical bone that has been processed to contain divets, holes, or both. The methods of this disclosure may be used to produce enriched tissue extracts from bone tissue. Moreover, the methods of this disclosure may also produce enriched tissue extracts that are specifically tailored to facilitate bone healing.

Cortical bone and cancellous bone may be obtained from a donor subject using standard techniques. Bone contains several inorganic mineral components, such as calcium phosphate, calcium carbonate, magnesium, fluoride, sodium, and the like. The mineral or calcium content of bone tissue obtained from a donor may vary. In some cases, cortical bone obtained from a donor may be about 95% mineralized, while cancellous bone may be about 35-45% mineralized. In some cases, cortical bone obtained from a donor may be about 73.2 wt % mineral content, while cancellous bone may be about 71.5 wt % mineral content. In some cases, the mineral content of bone tissue obtained from a donor is about 25% prior to demineralization. Additional information regarding the mineral content of bone and issues relating to demineralization can be found in U.S. Pat. No. 9,289,452, which is incorporated herein by reference in its entirety.

In some instances, the tissue biological sample may be or include tendon tissue. Exemplary tendon tissue includes semitendinosus, gracilis, tibialis, peroneus longus, and Achilles tendon Tendons are defined as flexible but inelastic cords of strong fibrous collagen tissue that attach muscle to bone. Tendons can be structured as single stranded, double stranded, double bundled, or in other pre-shaped configurations. Enriched tissue extracts produced from tendon tissue may be used as therapeutic compositions to treat a variety of medical conditions, including the treatment of, among others, tendon tears, ligament tears, muscle tears, bone fractures, wounds to the skin, and to assist in spinal fusion procedures.

In some instances, the tissue biological sample may include ligament tissue. Exemplary ligament tissues include patella ligament, knee cruciate ligaments, and spinal ligaments (e.g., ligamentum flavum). While the term ligament and tendon are often used interchangeability, ligaments attach bone to bone, whereas tendon attaches muscle to bone. Ligaments are defined as flexible but inelastic cords of strong fibrous collagen tissue that attach bone to bone. Ligaments can be structured as single stranded, double stranded, double bundled, or in other pre-shaped configurations. Enriched tissue extracts produced from ligament tissue may be used as therapeutic compositions to treat a variety of medical conditions, including the treatment of, among others, ligament tears, tendon tears, muscle tears, bone fractures, wounds to the skin, and to assist in spinal fusion procedures.

In some instances, the tissue biological sample may be or include periosteum tissue. Periosteum tissue is a dense irregular connective tissue that covers as a membrane the outer surface of all bones except at the joints of long bones. Periosteum tissue is a dense irregular connective tissue. Enriched tissue extracts produced from the periosteum tissue may be used as therapeutic compositions to treat a variety of medical conditions, including the treatment of, among others, bone injury, cartilage injury, osteochondral tissue injury, and to assist in spinal fusion procedures.

In some instances, the tissue biological sample may be or include skin tissue. Skin tissue is the thin outer layer of tissue on the human body. Skin has three layers: the epidermis, the dermis, and the hypodermis. The epidermis is the outermost waterproof layer; the dermis contains tough connective tissue, hair follicles, and sweat glands; and the hypodermis is the deeper subcutaneous tissue made of fat and connective tissue. Skin can be processed as either full-thickness skin or partial-thickness skin, depending on whether it includes the fat component of the hypodermis or just the outermost skin components. Partial-thickness skin contains the epidermal layer and a thin layer of dermis. It may be recovered from a donor with a dermatome, the recovery of which sets the overall thickness of the recovered partial-thickness skin. In some instances, full-thickness skin may have a thickness of about 1 mm to 5 mm. In some instances, partial-thickness skin may have a thickness of about 0.2 mm to 2 mm. Enriched tissue extracts produced from the dermal tissue may be used as therapeutic compositions to treat a variety of medical conditions, including the treatment of, among others, dermal scaring, dermatitis, skin grafts, and dermal burns, wound healing, and as a prophylactic for surgical dehiscence.

In some instances, the tissue biological sample may be or include cartilage tissue. Cartilage is flexible but inelastic cords of strong fibrous collagen tissue that cushions bones at joints and makes up other parts of the body. Cartilage tissue can be found throughout the human and animal anatomy (e.g., at joints, at the ends of ribs, between spinal vertebrae, and in the cars, nose, and throat). The cells within cartilage tissue are called chondrocytes. These cells generate proteins, such as collagen, proteoglycan, and elastin, that are involved in the formation and maintenance of the cartilage. Hyaline cartilage is present on certain bone surfaces, where it is commonly referred to as articular cartilage. In some instances, the tissue may be articular cartilage. Articular cartilage contains significant amounts of collagen (about two-thirds of the dry weight of articular cartilage), and cross-linking of the collagen imparts a high material strength and firmness to the tissue. These mechanical properties are important to the proper performance of the articular cartilage within the body. Additional information about cartilage tissue can be found in U.S. Pat. Nos. 9,186,380 and 9,186,253 and U.S. Application Publication Nos. 2017/0035937 and 2019/0166827, which are each incorporated herein by reference in their entireties. In some instances, tissue products as described therein may be processed according to the provided methods. In some instances, viability of native chondrocytes in the processed cartilage tissue may be important for utility of cartilage grafts made therefrom. Articular cartilage is not vascularized and, when damaged (such as by trauma or degenerative causes), has little or no capacity for in vivo self-repair. Enriched extracts from cartilage tissue comprising viable native chondrocytes may facilitate healing of such damage upon administration at a wound site by stimulating chondrogenesis in situ at the wound site.

In some instances, the tissue biological sample may be or include osteochondral tissue comprising bone tissue with a layer of cartilage tissue adhered thereto. For example, the osteochondral tissue can comprise osteochondral tissue from the humerus (e.g., humeral head), femur (e.g., femoral condyle), tibia, ilium, fibula, radius, ulna, trochlea, patella, talus, or ankle. Additional information about osteochondral tissue can be found in U.S. Pat. No. 9,168,140 and U.S. Application Publication Nos. 2017/0035937 and 2019/0166827, which are each incorporated herein by reference in their entireties. In some instances, tissue products as described therein may be processed according to the provided methods.

In some instances, the tissue biological sample may be or include fascia tissue. Fascia is layers of fibrous material within the body that surround muscles and other anatomical features. For example, an abundance of fascia connective tissue can be found at the quadriceps and inner or frontal thigh areas. Typically, fascia is flexible and contains collagen fibers which have been formed by fibroblasts. Embodiments of the present disclosure encompass techniques for enriched tissue extracts from fascia, processing the enriched tissue extracts into therapeutic products, and administering such products to recipient patients. Additional information about fascia can be found in U.S. Pat. No. 9,446,077, which is incorporated herein by reference. In some instances, tissue products as described therein may be processed according to the provided methods. Enriched tissue extracts produced from the fascia tissue may be used as therapeutic compositions to treat a variety of medical conditions, including the treatment of, among others, muscle tears or volumetric muscle loss.

In some instances, the tissue biological sample may include be or muscle tissue. Muscle is a band or bundle of fibrous tissue that has the ability to contract. Muscle tissue can be processed according to the present disclosure to produce an enriched tissue extract that is used as a therapeutic product to treat a variety of medical conditions.

In some instances, the tissue biological sample be or include neural tissue from nerves. Nerves bundles of fibers that use electrical and chemical signals to transmit sensory and motor information from one body part to another. Nerves can be processed according to the present disclosure to produce an enriched tissue extract that is used as a therapeutic product to treat a variety of medical conditions, including the treatment of, among others, brain injury, spinal cord injury, spinal nerve damage, peripheral nerve damage or loss, volumetric muscle loss.

In some instances, the tissue biological sample may be or include vascular tissue. Vascular tissue are tissue vessels that transport nutrients, such as veins, arteries, and capillaries. Vascular tissue can be processed according to the present disclosure to produce an enriched tissue extract that is used as a therapeutic product to treat a variety of medical conditions, including the treatment of, among others, lung transplants, lobectomies, and lung trauma.

In some instances, the tissue biological sample may be or include birth tissue. Birth tissue may include the amniotic sac (which includes two tissue layers, the amnion and chorion), the placenta, the umbilical cord, and the cells or fluid contained in each. Additional information about birth tissue can be found in U.S. Pat. Nos. 9,358,320 and 9,480,549, which are each incorporated herein by reference in their entireties. In some instances, tissue products as described therein may be processed according to the provided methods. Amnion is the innermost layer of the placental membranes. It is a thin semi-transparent membrane normally 20 μm to 500 μm in thickness. The amnion comprises a single layer of ectodermally derived columnar epithelial cells adhered to a membrane comprised of collagen I, collagen III, collagen IV, laminin, and fibronectin which in turn is attached to an underlying layer of connective tissue. The connective tissue includes an acellular compact layer of reticular fibers, a fibroblast layer, and a spongy layer consisting of a network of fine fibrils surrounded by mucus. The thicker chorion tissue contains all of the vascular vessels and capillaries, nerves and majority of the cells, although a single layer of specialized epithelial cells line the inner-most surface of the amnion tissue (the side closest to the baby). Amniotic membrane has been used for many years in various surgical procedures where anti-scar formation is desired such as, for example, treatment of skin, ocular surface, spine, knee, child birth-related injuries, shoulder surgery, spinal surgeries, trauma related cases, cardiovascular procedures, brain/neurological procedures, burn and wound care, etc. The enriched tissue extract made from the amniotic membrane may be used as a therapeutic product for healing in these cases or injuries. These enriched tissue extracts may provide good wound protection, can reduce pain, reduce wound dehydration, increase cellular healing or proliferation, and provide anti-inflammatory and antimicrobial effects.

In some instances, the tissue biological sample may be or include adipose tissue. Adipose tissue is a loose connective tissue comprises adipocytes which is located throughout the body, including under the skin and in deposits between the muscles and around organs. Besides adipocytes, adipose tissue contains connective tissue matrix, nerve tissue, stromovascular cells, and immune cells. Together these components function as an integrated unit. Additional information about adipose tissue can be found in U.S. Patent Publication Nos. 2014/0056865 and 2017/0035937, which are each incorporated herein by reference. In some instances, tissue products as described therein may be processed according to the provided methods. In some instances, the biological sample may be a stromal vascular fraction of adipose tissue. Information about stromal vascular fraction can be found in U.S. Pat. No. 10,568,990 and U.S. Patent Publication No. 2017/0035937, which are incorporated herein by reference. Adipose tissue and adipose-derived stromal vascular fraction used in the methods provided in this disclosure may produce enriched tissue extracts that can be used to treat a variety of medical conditions, including the treatment of, among others, ailments requiring a correction of age-, surgery-, and disease-related facial depressions and rhytids (wrinkles) and other conditions that require volume augmentation at other body sites.

### 2. Isolated Cells

In some embodiments of the present disclosure, the biological sample may be or include isolated cells (i.e. cells that are not contained within the structure of a tissue). In some embodiments, the biological sample may be or include isolated primary cells. In some embodiments, the biological sample may be or include immortalized cells. In some embodiments, the biological sample may be or include genetically modified cells.

Primary cells are cells that have been isolated directly from human or animal tissue using enzymatic or mechanical methods. Once isolated, they are placed in an artificial environment in plastic or glass containers supported with specialized medium containing essential nutrients and growth factors to support proliferation. Primary cells can be of two types—adherent or suspension. Adherent cells require attachment for growth and are referred to as anchorage-dependent cells. The adherent cells are usually derived from tissues of organs. Suspension cells do not require attachment for growth and are referred to as anchorage-independent cells. Some tissue-derived suspension cells are available such as hepatocytes or intestinal cells. Primary cells usually have a limited lifespan. Primary cells can include cells isolated from any of the tissue types described in this disclosure that can be used as tissue biological samples. In some instances, the isolated primary cells may include mesenchymal stem cells. Mesenchymal stem cells are multipotent stromal cells that can differentiate into a variety of cell types, including osteoblasts (bone cells), chondrocytes (cartilage cells), myocytes (muscle cells), and adipocytes (fat cells). In some cases, the isolated primary cells are substantially free from other cell types (i.e. are population of cells of the same type).

Immortalized cells are defined as a population of cells that, due to mutation or artificial modification, can escape normal cell senescence and continue cellular division. These types of cells can grow in vitro for prolonged periods and are often utilized in cell cultures due to their extended life span. Immortalized cells can be grown from a specific cell line or may be commercially purchased. Exemplary immortalized cells include Jurkat cells and immortalized tenocytes. In some instances, the immortalized cells may be tailored to a specific cell line, such as Jurkat cells, which are an immortalized line of human T lymphocyte cell, or immortalized tenocytes, which are immortalized cells from Achilles tendon. In other cases, the immortalized cells may be undetermined and multipotent. For example, in some embodiments, the isolated immortalized cells may be immortalized mesenchymal stem cells.

Genetically modified cells are cells that have a genetic modification to the cell genome such that expression of one or more proteins is altered or introduced. In some instances, the genetically modified cells are genetically modified mesenchymal stem cells such as genetically modified immortalized mesenchymal stem cells. For example, the genetically modified cells may have a genetic modification that results in expression of at least one recombinant growth factor that is not normally expressed by the cells. In some cases, the genetically modified cells may have a genetic modification that results in overexpression a growth factor that is normally expressed by the mesenchymal stem cells. The expression or overexpression of a growth factor by the genetically modified cells may be tailored depending on the desired healing effects of the enriched tissue extract produced from the genetically modified cells and/or the type of injured tissue to be treated.

### 3. Preparation of Biological Sample

The biological sample may be prepared in various ways before being used to produce an enriched tissue extract. For example, in some instances, the biological sample may be cleaned first. In some embodiments, the cleaning is performed using conventional cleaning techniques, such as the standard cleaning protocol of the American Association of Tissue Banks (AATB). Other conventional methods of cleaning tissue or tissue graft products may also be used. In some instances, the biological sample may be cleaned using systems and methods as described in U.S. Pat. Nos. 7,658,888, 7,776,291, 7,794,653, 7,919,043, 8,303,898, and 8,486,344, each of which are incorporated herein by reference in their entireties. According to some embodiments, it may be desirable to clean the biological sample to remove blood and other liquids and/or particulates before using the biological sample to produce an enriched tissue extract.

In instances where the biological sample is a tissue biological sample, cleaning of the tissue biological sample may include removing tissue so as to result in a tissue biological sample that is substantially a single type of tissue and is substantially free of other types of tissue types.

In some instances, the biological sample may be maintained in a fresh state at refrigerated temperatures so as to retain the viability of cells therein.

In some instances, the biological sample may be processed to kill all or substantially all cells present in the sample. Death of or killing all or substantially all cells in the biological sample refers to death of 90-100% of the cells in the biological sample. Common methods of cell lysis that are known in the art are homogenization, freeze/thaw treatments, extreme heat treatments, and osmotic or chemical lysis. Some of these methods are also discussed elsewhere in this disclosure as methods of stimulating a biological sample by applying a physical stress to the biological sample. However, depending on the conditions chosen for these methods, disruption of all or substantially all of the cells in a biological sample may be achieved. The application of stress to the biological sample may be used to disrupt the structural integrity of the cells within the biological sample, thereby inducing excretion or expulsion of the inner cellular material, which contains biologically active components produced by the cells. The inner cellular material may include the biologically active components that the cells, when alive, would have secreted in response to injury to stimulate healing. For example, in some instances, the method of causing death of the cells in the biological sample induces a stress response in the cells prior to their death such that biologically active components are produced in the cells that are subsequently excreted or expelled from the cells upon cell lysis.

Lysis may be achieved by extensive homogenization of the biological tissue. Homogenization can include any of mechanical homogenization, ultrasonic homogenization, resonant acoustic homogenization, and pressure homogenization. Mechanical homogenization relies on the use of handheld or motorized devices with rotating blades in breaking down and extracting proteins. The tangential force applied by the blades to the sample facilitates the disruption of the cell wall and subsequent homogenization of the sample. This method is most suitable when working with soft, solid tissues.

Ultrasonic homogenization (cavitation) involves the use of an acoustic transducer to deliver high-frequency sound waves to biological samples in solution such as liquid cell suspensions. The mechanical energy produced in the process facilitates the formation of microscopic bubbles which then cause shock waves to radiate throughout the sample once they implode. This method can be used to homogenize small batches (less than 100 ml) of cell and finely diced tissue samples.

Low frequency, high-intensity acoustic energy may be used to create a uniform shear field throughout the entire processing vessel, which results in rapid fluidization (like a fluidized bed) and dispersion of material. The biological sample is placed in a processing vessel together with a processing solution such as saline, a buffered solution, or a cell culture medium and then placed into a resonant acoustic vibration device which introduces acoustic energy to the processing vessel, and the biological sample and processing solution therein. In some instances, the resonant acoustic vibration device includes an oscillating mechanical driver that create motion in a mechanical system comprised of engineered plates, eccentric weights and springs. The energy generated by the device is then acoustically transferred to the material to be mixed. The underlying technology principle of the resonant acoustic vibration device is that it operates at resonance. An exemplary resonant acoustic vibration device is a Resodyn LabRAM ResonantAcousticR Mixer (Resodyn Acoustic Mixers, Inc., Butte, Montana). In some instances, the resonant acoustic vibration device may be devices such as those described in U.S. Pat. No. 7,866,878 and U.S. Patent Application No. 2015/0146496, which are each incorporated herein in their entireties. Resonant acoustic energy may be applied to a biological sample in solution using the equipment and methods similar to those as described in U.S. Patent Publication Nos. 2017/0035937 and 2018/0280575, which are each incorporated herein by reference in its entirety, the parameters of such methods selected such that complete or substantial lysis of the cells within the biological sample occurs.

In high-pressure homogenization, a biological sample is forced through a narrow space while applying pressure into the sample. Extracting proteins in higher pressures (40 and 80 MPa) can significantly increase or even double the protein recovery rate. High-pressure homogenizers are also scalable and so can be adapted to different sample sizes.

Cell lysis may be achieved by freezing or cryofracturing the biological sample. For example, the biological sample may be frozen and then thawed before using the biological sample to produce an enriched tissue extract. Cryofracturing may be particularly useful for tendon tissue or cartilage tissue. Cryofracturing the biological sample generally includes freezing and then macerating the biological sample. In one example, the biological sample is segmented into thin strips before being introduced to liquid nitrogen cooled grinder. A solid carbon dioxide cooled grinder or any other means of cryofreezing the biological sample may be used. The cryofrozen biological sample may then be macerated or otherwise fractured or broken down. In some cases, fracturing the cryofrozen biological sample may include milling or grinding the biological sample. Exemplary cryofracturing techniques are disclosed in U.S. Pat. No. 9,162,011, which is incorporated herein by reference. Conditions that are well-known in the art may be chosen such that all or a substantial portion of the cells within the biological sample may be lysed. Generally, the biological sample is frozen in the absence of a cryopreservative agent (e.g., DMSO, glycerol, sucrose).

Heat can likewise be used to achieve cell lysis, with high temperatures causing damage the membrane by denaturizing the membrane proteins and results in the release of intracellular organelles. Temperatures in excess of 50° C., 60° C., 70° C., 80° C. can be used. In some instances, samples may be pretreated with a lysozyme to facilitate cell membrane disruption as well.

In some embodiments, the biological sample may be or include bone tissue, and the bone tissue may be demineralized prior to using it to produce an enriched tissue extract. Demineralization of bone typically kills all or substantially all of the cells native to the processed bone tissue, removing cellular matter from the demineralized bone. Methods of demineralizing bone are well known in the art. Exemplary methods are described in U.S. Pat. Nos. 9,192,695 and 9,289,452 and U.S. Patent Publication No. 2017/0035937, which are incorporated herein by reference in their entireties. In some instances, the mineral content of the demineralized bone tissue may less than 20% (e.g., less than 18%, less than 15%, less than 10%, or less than 8%) such as, for example, 1-7%.

The biological sample may also be subjected to osmotic shock to kill all or substantially all cells present in the sample. When the concentration of salt surrounding a cell is suddenly changed such that there is a concentration difference between the inside and outside of the cell, the cell membrane becomes permeable to water due to osmosis. If the concentration of salt is lower in the surrounding solution, water enters the cell and the cell swells up and subsequently bursts.

Chemical lysis methods use lysis buffers to disrupt the cell membrane. Lysis buffers break the cell membrane by changing the pH. Detergents can also be added to cell lysis buffers to solubilize the membrane proteins and to rupture the cell membrane to release its contents. Chemical lysis can be classified as alkaline lysis and detergent lysis.

In alkaline lysis, OH ions are the main component used for lysing cell membrane. The lysis buffer consists of sodium hydroxide and sodium dodecyl sulphate (SDS). The OH″ ion reacts with the cell membrane and breaks the fatty acid-glycerol ester bonds and subsequently makes the cell membrane permeable and the SDS solubilizes the proteins and the membrane. The pH range of 11.5-12.5 is preferable for cell lysis. Although this method is suitable for all kinds of cells, this process is very slow and takes about 6 to 12 hours.

In some instances, detergent may be used. Detergents also called surfactants have an ability to disrupt the hydrophobic-hydrophilic interactions. Since the cell membrane is a bi-lipid layer made of both hydrophobic and hydrophilic molecules, detergents can be used to disintegrate them. Detergents are capable of disrupting the lipid-lipid, lipid-protein and protein-protein interactions. Based on their charge carrying capacity, they can be divided into cationic, anionic and non-ionic detergents.

Non-ionic detergents may be preferred as they cause the least amount of damage to proteins and enzymes. 3-[(3-cholamidopropyl)dimethylammonio]-1-propanesulfonate (CHAPS) and 3-[(3-cholamidopropyl)dimethylammonio]-2-hydroxy-1-propanesulfonate (CHAPSO), a zwitterionic detergent, are commonly used non-ionic detergents. Other non-ionic detergents include Triton-X™ and Tween™ series. Ionic detergent such as sodium dodecyl sulphate (SDS) is widely used for lysing cells but may result in high amount of protein denaturation. It is used in gel electrophoresis and western blotting techniques. Chaotropic agents can also be used for cell lysis. These include urea, guanidine and Ethylenediaminetetraacetic acid (EDTA) which can break the structure of water and make it less hydrophilic and there by weakening the hydrophobic interactions. An additional purification step may be required in the preparation of the biological sample if the cell lysis protocol uses detergents.

### B. Stimulating Through Physical Stress

In some embodiments, the methods may include a step of stimulating the biological sample. A stimulation step may be included in the method where the biological sample includes live cells, which may respond to the stimulation. In particular, methods for producing an enriched tissue extract from a biological sample that includes live cells may include administering a physical stress on the biological sample. In some instances, the application of stress on the biological sample may induce a stress response in the live cells of the biological sample, thereby inducing the live cells to release biologically active components in response. The biologically active components may be the natural response of the cells to injury to stimulate healing of the cells and nearby tissue. Without being held to any particular theory, the stress response of the live cells of the biological sample may result in the production and/or secretion of biologically active components that are similar in kind and/or amount to those produced and/or secreted by the same kind/type of cell in the body in response to injury to promote healing. The enriched tissue extracts produced via the methods described herein may include one or more of these biologically active components, such as for example, micro-vesicles, exosomes, growth factor proteins, nucleic acids, extracellular matrix proteins, or cytokines. Signaling molecules may include one or more amino acids, hormones, cytokines, neurotransmitters, cyclic AMP, or steroids.

A variety of physical stresses may be used to induce the stress response in the live cells of the biological sample. For example, the physical stress may include one or more of a mechanical stress, a chemical stress, or a temperature stress, particularly a hypothermic stress. Different types of physical stress may induce a different stress response from the live cells, and may, in some cases, impact the type and amount of biologically active components produced by the live cells. As discussed elsewhere in this disclosure, the application of stress to the biological sample may disrupt the structural integrity of at least some of the cells within the biological sample, thereby inducing excretion or expulsion of biologically active components from the cells. However, when the methods discussed below are used to stimulate cells to produce a stress response, conditions are chosen so that all or substantially all of the cells within a biological sample remain viable (i.e. alive). In instances where biological samples are prepared that contain no or substantially no live cells, such samples do not require performance of a stimulation step.

In some embodiments, no physical stress may be required to cause the live cells to produce and/or release biologically active components from the biological sample. Thus, according to those embodiments, the methods provided herein may not include administering a stimulation step to the biological sample because the live cells are able to release biologically active components without requiring induction of a stress response or disruption of the structural integrity of the cells.

### 1. Mechanical Stress

In one example, administering physical stress on the biological sample may include administering mechanical stress on the biological sample. Mechanical stress may include applying force on the biological sample for a specified duration of time. For example, the biological sample may be exposed to or subjected to physical stress or strain. In some instances, the biological sample may be exposed to sinusoidal tension or compression to induce a stress response from the live cells. For example, various responses from live cells resident in the tissue might be induced by different mechanical loading regimens such as super-physiological strain (e.g., 30% and greater, depending on the tissue) or repetitive and/or uninterrupted strain to simulate a chronic condition.

Depending on the intensity (e.g., amount of force, tension, speed, frequency, duration, etc.) of the mechanical stress applied to the biological sample, some amount of cell lysis may occur. Some amount of cell lysis may be desirable in some cases to adequately simulate tissue injury or disease. However, in other instances cell lysis may be undesirable. In such cases, the intensity of the mechanical stress may be adjusted to induce the stress response from the live cell to cause minimal cell lysis such that 90-100% of the cells of the biological sample remain alive (for example, less than 10%, 9%, 8%, 7%, 6%, 5%, 4%, 3%, 2%, or 1% of cells in biological sample die) or only some amount of cell lysis such that at least 70% of the cells of the biological sample remain (for example, up to 30%, up to 20%, up to 10%, 10-30%, 10-20%, 20-30%, 10-15%, 15-25%, 10-25%, or 15-30% of the total cells in the biological sample die).

One way of inducing mechanical stress may include mechanically loading the biological sample. Mechanically loading the biological sample can induce a stress response in the cells within in the biological sample. For example, mechanically loading the biological sample can influence secretions, growth factors, signaling, and cellular differentiation. Exemplary methods and concepts relating to mechanically loading of a biological sample are described in HALL, et al., “Paralysis and Growth of the Musculoskeletal System in the Embryonic Chick”, Journal of Morphology, Vol 206, Issue 1, pp 45-56, October 1990 and MAEDA, et al., “Conversion of Mechanical Force into TGF-β-Mediated Biochemical Signals”, Current Biology, Vol 21, No 11, pp. 933-941, Jun. 7, 2021. Mechanically loading the biological sample may include applying compressive loads to the biological sample and/or applying static and dynamic tensile loads to the biological sample. In some instances, mechanically loading may include mechanically stretching the biological sample. Mechanically stretching tissue is known to effect cellular functionality and organization. For example, mechanical stretching has been shown to impact cell morphology, proliferation, lineage commitment, and cellular differentiation. Exemplary methods and concepts relating to mechanical stretching of live cells are described in Riehl, B. D., et al., Mechanical stretching for tissue engineering: two-dimensional and three-dimensional constructs. Tissue Eng Part B Rev. 18(4):288-300 (2012). The stress response to mechanical stretching may vary depending on cell type and the type of loading mode. In some embodiments, a customized bioreactor or a materials testing machine may be used for mechanically loading the biological sample. An exemplary materials testing machine for applying a mechanical load to tissues is a Universal Testing Machine by Instron®. The modes, frequencies, amplitudes and patterns of the mechanical loading may be varied depending on the desired effect on the cells. For example, different secretory profiles of the cells within the biological sample may be induced by varying the mode, frequency, amplitude, and/or pattern of mechanical load applied to the biological sample.

Mechanical stress can be applied on a tissue biological sample, such as muscle tissue or tendon tissue, in a similar manner as is done in methods for performing fatigue testing. Fatigue testing is a specialized form of mechanical testing that is performed by applying cyclic loading to a coupon or structure, which can be used to generate fatigue life and crack growth data, identify critical locations or demonstrate the safety of a structure that may be susceptible to fatigue. Exemplary methods and concepts relating to mechanically stressing live cells are described in Shepherd, J., & Screen, H. et. al. Fatigue loading of tendon. International Journal of Experimental Pathology, 94(4), 260-270 (2013). For example, a tissue biological sample may be cycled to a constant peak load and increase in extension monitored may be performed as is done in creep analysis. Creep behavior can be described by three stages of deformation. An initial primary stage associated with rapid extension is followed by a relatively stable secondary stage in which there is a steady increase in sample length, followed by a tertiary stage, as the sample rapidly extends to rupture. In another example, loading of a tissue biological sample may be carried out to a constant peak displacement and the reduction in load considered as is done in stress relaxation analysis. Stress relaxation curves tend to follow an exponential curve, with stress steadily stabilizing after an initial rapid decrease. With different boundary conditions, these methods are expected to elicit a different response from the loaded tissue biological sample, but are often used interchangeably. While stress relaxation tests will not terminate in the failure of the sample, fatigue damage is generated; this has been confirmed by stopping tests and loading samples to failure, where significant decreases in quasi-static mechanical characteristics are reported (e.g., for tendon tissue samples in Legerlotz et al., 2011, Scand. J. Med. Sci. Sports 23:31-37).

In some instances where the biological sample comprises isolated cells, the mechanical stress may include growing the cells in a pressurized vessel or otherwise applying pressure to the cultured cells. For example, mechanical stress may be applied via a mechanical bioreactor that mechanically stimulates cells in culture by the application of direct tension and compression mechanical load. An exemplary mechanical bioreactor is a TC-3 Bioreactor by EBERS Medical Technology. Typical loading regimens might include tensile or compressive strain from 1 to 10% and frequencies from 0.1 to 10 Hz, though parameters outside these ranges might be suitable depending on the cell type, scaffold type and particular goals of the mechanical stimulation. In some embodiments, the biological sample may be subjected to pressurization in a cyclical manner (i.e. one or more periods of pressurization interspersed with periods where the biological sample is not subjected to pressurization).

Another way of inducing mechanical stress may include subjecting the biological sample to homogenization. For example the biological sample may be blenderized, ground, macerated, and/or pureed in a blender, a ball mill, or other grinding device. For example, a ball mill may be used to fragment or grind a biological sample. The movement of the material and the one or more grinding components in the ball mill processing vessel results in fragmentation of the tissue. In another example, a tobacco grinder may be used to grind the biological sample into fine filaments. The stresses applied by these devices break apart larger pieces of tissue and may disrupt cell membranes as well as exerting force onto the biological sample. Homogenizing the biological sample may induce a stress response in any living cells present in the biological sample and/or may disrupt the cellular structure integrity of the cells. The disruption of the extracellular matrix components can expose or otherwise make available to cells the presence of signaling molecules resident but previously latent in the matrix. Additionally, the disruption of the organized extracellular matrix can relieve the physiological stresses that cells normally may experience and consequently induce cell responses through changes in mechanotransduction signaling pathways. In some embodiments, the stresses applied by the ball mill or blender may disrupt the structural integrity of the cellular structure, exposing or causing expulsion of cellular material internal to the cells. For example, when the biological sample includes bone tissue, grinding the biological sample may be a preferred means of applying mechanical stress. In some instances, a ball mill processing vessel and associated ball milling methods as described in U.S. Patent Publication No. 2018/0280575, which is incorporated herein by reference, may be used to induce mechanical stress according to the provided methods.

Another way of applying mechanical stress on the biological sample may include tenderizing the biological sample (i.e. mechanical impacts) so as to mimic blunt force trauma. A tenderizer tool may be a mallet-type tool (or the like) or a blade tenderizer tool, for example, having a series of blades or nails designed to puncture the tissue and cut into fibers thereof. The tenderizer may break down the tissue, causing disruption of the cellular structure. Tenderizing can be used to apply mechanical stress to a variety of soft tissues including but not limited to dermal tissue.

Mechanical stress can also be applied to the biological sample using resonant acoustic frequencies that are matched to the harmonic frequency of the live cells within the biological sample or to physically vibrate the biological sample to induce a stress response. Application of resonant acoustic frequencies includes applying a resonant frequency to the biological sample. The resonant frequency is selected to match the harmonic frequency of the biological sample and causes the live cells to resonate. In some instances, the applied frequency to achieve resonance is in the range of 55-65 Hz. Causing the live cells to resonate may focus the energy input by the resonant acoustic frequency within the live cells, resulting in mechanical stress. The conditions (e.g., frequency, duration, amplitude, energy input, etc.) of the resonant acoustic frequencies applied may be selected to induce stress on the live cells in the biological sample. For example, the amplitude (maximum displacement) applied to the biological sample may be adjusted depending on the biological sample to achieve a desired mechanical stress. In some instances, the amplitude applied to the biological sample is in the range of 0.01-0.50 inches. In some instances, the intensity or acceleration of the resonant acoustic frequencies applied to the biological sample (i.e. the level of energy input to the biological sample) may be adjusted depending on the biological sample to achieve a desired mechanical stress. The level of energy input applied to the biological sample can be measured in units of acceleration of gravity, ‘g.’ In some instances, the energy input applied to the biological sample is in the range of 0-100 g-force. In some instances, adjusting the energy input is equivalent to changing the amplitude of the resonant acoustic frequencies applied to the biological sample. The greater the amplitude (distance traveled) the greater the acceleration (g-force). In some instances, resonant acoustic energy may be applied to a biological sample using the equipment and methods similar to those as described in U.S. Patent Publication Nos. 2017/0035937 and 2018/0280575, which are each incorporated herein by reference in its entirety, the parameters of such methods selected such that physical stress of the live cells within the biological sample occurs.

### 2. Chemical Stress

In some instances, administering physical stress may include subjecting the biological sample to chemical stress. In some embodiments, the chemical stress may include contacting the biological sample with a hypertonic solution or hypotonic solution. Depending on the state of the live cells in the biological sample, the hypertonic solution or hypotonic solution may induce osmotic stress on the live cells. High or low osmotic stress may be induced on the live cell, depending on the solution used. Osmotic stress may exert physical strain on the cell walls and thereby induce a stress response from the live cells. In some cases, osmotic stress may destabilize or rupture cell membranes of at least some of the live cells. Examples of solutions that could be used include hypotonic (≤0.9%) or hypertonic (>1%) sodium chloride. The biological sample may be exposed to a hypertonic solution or hypotonic solution for a time period from 2 minutes to 24 hours (e.g., 5 minutes to 20 hours, from 15 minutes to 18 hours, from 30 minutes to 12 hours, from 1 hour to 6 hours, or from 2 hours to 4 hours).

In some embodiments, administering chemical stress on the biological sample may include exposing the biological sample to an environment that is not at physiological pH. Living tissue and cells thrive under homeostatic conditions that include a very narrow pH range. For example, the physiological pH for humans is approximately 7.4. Thus, when the cellular environment is changed, such as becoming more acidic or basic, live cells will respond in an effort to regain homeostasis such as, for example, producing and, in some instances, secreting biologically active components that impact the acidity or alkalinity of their environment back to homeostatic levels. For example, if the biological sample is introduced to a highly acidic solution (high concentration of H+), the live cells may be exposed to a low pH environment. Hydrogen ions are very reactive and can react with cellular components resulting in denaturation of proteins and disruption of cell membranes. In response to the low pH environment, the live cells may secrete biologically active components to prevent reaction with the acidic solution or to attempt repairs of the damage caused by the acidic solution. In some instances, administering this type of chemical stress on the biological sample may can include incubating the biological sample in a growth medium devoid of sodium bicarbonate for a period of hours.

For example, in some instances, the biological sample may be exposed to an acidic solution. An acidic solutions may include a solution that contains hydrochloric acid (HCl), acetic acid (CH₃COOH), citric acid (C₆H₈O₇), formic acid (CH₂O₂), ethylenediaminetetraacetic acid (EDTA), nitric acid (HNO₃), propionic acid (C₃H₆O₂), phosphoric acid (H₃PO₄), gluconic acid (C₆H₁₂O₇), malic acid (C₄H₆O₅), tartaric acid (C₄H₆O₆), and fumaric acid (C₄H₄O₄). In some instances, the acid solution is a mineral acid. Mineral acids include, but are not limited to, hydrochloric acid (HCl), nitric acid (HNO₃), phosphoric acid (H₃PO₄), sulfuric acid (H₂SO₄), boric acid (H₃BO₃), hydrofluoric acid (HF), hydrobromic acid (HBr), perchloric acid (HClO₄), and hydroiodic acid (HI). In some instances, the acid solution may be ethylenediaminetetraacetic acid (EDTA).

Exemplary methods and concepts relating to chemically stressing live cells using changes in environmental pH are described in Taylor, A. Responses of Cells to pH Changes in the Medium. The Journal Of Cell Biology, 15(2): 201-209 (1962). In some instances, the biological sample (tissue or isolated cells) may be incubated in water or a saline solution. In some instances, a buffered solution may be used such as, for example, a carbon dioxide-bicarbonate buffer (e.g., Basal Medium Eagle) or a hydroxymethyl aminomethane (Tris) buffer. In some instances, the pH of the solution can be adjusted with HCl and NaOH. In some instances, when using a carbon dioxide-bicarbonate buffer system, the pH may be adjusted by changing increasing or decreasing the concentration of NaHCO₃ in the buffer solution (while maintaining osmotic balance of the solution by making corresponding adjustments to the concentration of other salts (e.g., NaCl) in the solution) and incubating the biological sample in the solution in atmospheric conditions with varying concentrations of CO₂ (e.g., from 0.5% to 80% mixed with air). Using such methods, raising or lowering the pH as little as 0.2 of a pH unit may induce a response from the live cells. In some cases, normal cellular activity may be regained after homeostasis is regained. For example, live cells kept at a pH of 6.5 for an hour may recover without apparent damage. However, when the pH reaches 8 or above, cells may undergo contraction, detachment, or disruption of the cellular integrity. When the pH is reduced, for example from 7.3 to 5.6, cellular components may become immobilized and normal cellular processes may cease. In some embodiments, changing the pH may cause destabilization or rupturing of cell membranes of at least some of the live cells. Similar results may be achieved using a hydroxymethyl aminomethane (Tris) buffer or no buffer and adjusting the pH of the solution with HCl and NaOH.

In another embodiment, the biological sample may be exposed to hypo- or hyper-physiologic oxygen levels. Various tissues in the body experience differential physiological oxygen levels. Some organs function normally in vivo at oxygen levels ranging from 2-8%. However, in vitro culture conditions typically have an oxygen level of approximately 20%, within the typical range of normal atmospheric pressure (defined by OSHA as 20%-21%). By altering oxygen tension in a tissue culture environment, nearly every aspect of cell function can be affected. For example, varying the oxygen levels for cells (e.g., mesenchymal stem cells) can influence various cellular functions such as metabolism, differentiation capacity, proliferation, motility and genomic stability. Stress responses of live cells are known to be oxygen dependent. For example, cell secretion of various proteins or materials may be induced by altering oxygen tension. Exemplary methods and concepts relating to varying oxygen level exposure of live cells are described in MAS-BARGUES, et al., “Relevance of Oxygen Concentration in Stem Cell Culture for Regenerative Medicine” Review, International Journal of Molecular Sciences, 27 pages, Mar. 8, 2019. By altering the oxygen exposure of a biological sample, the tissue extracts produced may vary according to oxygen levels and thus result in different extracts, each with unique properties. Depending on the desired effect on the biological sample, the biological sample may be exposed to various oxygen levels, including hypoxic and hyperoxic levels. For example, the biologic sample can be exposed to oxygen levels of 0.5-30% in an incubator or bioreactor environment such as, for example, 0.5%, 1%, 2%, 5%, 8%, 10%, 12%, 15%, 18%, 20%, 22%, 25%, 28%, or 30%. In some embodiments, the biological sample may be exposed to hypoxic oxygen levels of 0-20%, 0-15%, 0-10%, 0-5%, or less than 5%. In other embodiments, the biological sample is exposed to hyperoxic levels of 20-30%, 25-30%, or more than 30%. In some instances, the biological sample is exposed to an oxygen level of 1.0% to ambient level (atmospheric; variable based on natural environment and altitude), such as, for example, 1%, 2%, 5%, 8%, 10%, 12%, 15%, 18%, or 20%.

### 3. Temperature Stress

In some instances, administering physical stress on the biological sample may include subjecting the biological sample to one or more temperature stress. In particular, the temperature stress mimics a hypothermic stress within the live cells of the biological sample. For example, a temperature stress may include subjecting the biological sample to temperatures such as 37° ° C. to 0° C. or −40° ° C. to 0° C. such as, for example, −20° C. to 0° C., for a limited period of time so as to induce a hypothermic stress response in the cells of the biological sample but not result in substantial cell death. The biological sample would be maintained at a temperature of 0° C. or below for a period of time that would result in minimal cell death or only some cell death, which depends in part on the size of the biological sample (e.g., tissue biological sample). In some instances, the biological sample is placed directly at the desired end temperatures. In other instances, the biological sample be exposed to gradually reducing temperatures over time. Exposure to hypothermic temperatures may result in the formation of ice crystals within or around the live cells of the biological sample, which may disrupt cell membranes any other cellular structures. This disruption may induce the cells to have a stress response in which biologically active components are produced and may be secreted.

In some instances, the temperature stress may mimic hyperthermia stress within the live cells of the biological sample. For example, a temperatures stress may include heating the biological sample. For example, the biological sample may be heated at temperatures up to 40° ° C., 45° C., 50° C., 55° C., 60° C., 65° C., 70° C., 75° C., 80° C., 85° C., or 90° C. such as, for example, from 38° C. to 43° C., 38° C. to 70° C., from 38° C. to 60° C., from 38° C. to 50° ° C., from 45° C. to 70° C., from 45° ° C. to 60° ° C., from 45° C. to 50° C., from 50° C. to 70° C., or from 50° C. to 60° C. A temperature may be selected that results in stressing the live cells without or with minimal denaturing membrane proteins.

The biological sample would be maintained at a temperature above 37ºC for a period of time that would result in minimal cell death or only some cell death, which depends in part on the size of the biological sample (e.g., tissue biological sample). In some instances, the biological sample is placed directly at the desired end temperatures. In other instances, the biological sample is heated slowly overtime at a gradual reduction of temperature. The time duration of heating the biological sample may affect the stress response. In some cases, the time duration for heating the biological sample to induce temperature stress may be proportional to the temperature. For example, heating the biological sample to a higher temperature may be performed over a short duration of time to prevent denaturing of the membrane proteins. Heating the biological sample to a lower temperature may be performed over a long duration of time to induce a specific stress response or release of specific intracellular materials. In some instances, the time duration of heating the biological sample may depend on the type of cell or tissue of the biological sample.

Following heating, the biological sample may be quenched. Heating the biological sample may cause swelling within or around the live cells of the biological sample, which may disrupt cell membranes. This disruption may induce the cells to have a stress response in which biologically active components are produced and may be secreted.

### 4. Growth Factor Treatment

According to some embodiments of the present disclosure, the methods may include a step of treating the biological sample with a growth factor supplement either before and/or after the physical stress is applied. For example, the growth factor supplement may include one or more of transforming growth factor β (TGF β)-1, -2 or -3, fibroblast growth factors (FGF)-2 and -4, bone morphogenetic proteins (BMP)-2, -4, -7, -9, vascular endothelial growth factor, platelet-derived growth factor, and epidermal growth factor. In exemplary methods, the biological sample is treated with a growth factor supplement before application of the physical stress. Without being bound by theory, it is theorized that treatment of the biological sample with a growth factor supplement may moderate the stress response from the live cells.

In some instances, the biological sample may be stimulated with a growth factor supplement without any application of a physical stress. For example, in some embodiments, the methods for producing enriched tissue extracts may include treating the biological sample with a growth factor supplement and then incubating the biological sample to secrete the biologically active components from the live cells of the biological sample. In such cases, a stress response may not be needed for the live cells to secrete the biologically active components. Instead, the growth factor supplement may promotes secretion of the biologically active components.

### C. Extraction

### 1. Incubation

Following any of the described steps in Sections A and B, the biological sample may be incubated in an extraction solution to form an enriched tissue extract. The biological sample may be incubated in the extraction solution for a period of time sufficient for biologically active components to be extracted from the biological sample. The biologically active components may include one or more of micro-vesicles, exosomes, secretomes, growth factor proteins, cytokines, nucleic acids, extracellular matrix proteins, or signaling molecules. The signaling molecules may include amino acids, hormones, cytokines, neurotransmitters, cyclic AMP, or steroids. The biologically active components may be secreted from the live cells of in the biological sample or, cell lysis has occurred, may be present in the inner cellular material expelled or secreted from the cells and/or present in the extracellular matrix of the biological sample.

Incubation of the biological sample in the extraction solution may be done at a temperature to sustain the live cells in the biological sample and/or induce secretion of the biologically active components from the live cells. In some embodiments, the biological sample may be incubated at a temperature from 2° ° C. to 45° C., from 2° ° C. to 30° ° C., from 5° C. to 37° C., or from 5° ° C. to 38° C., from 2° ° C. to 8° C., from 2° ° C. to 15° C., from 10° C. to 20° C., from 20° ° C. to 30° C., from 30° ° C. to 40° C., from 20° ° C. to 40° C., from 30° C. to 38° ° C., from 31° C. to 33° C., from 35° ° C. to 38° C., or from 35° ° C. to 45° C.

The biological sample may be incubated in the extraction solution for a period of time sufficient for the biologically active components to be extracted from the biological sample. The period of time sufficient to extract to the biologically active components may vary, but may be from 1 minute to 60 minutes, 1 minute to 48 hours, from 2 minutes to 42 hours, from 2 minutes to 36 hours, from 5 minutes to 32 hours, from 5 minutes to 24 hours, from 10 minutes to 18 hours, from 15 minutes to 12 hours, from 30 minutes to 10 hours, from 1 hour to 8 hours, from 1 hour to 6 hours, from 1 hour to 12 hours, from 3 hours to 10 hours, or from 6 hours to 8 hours.

In some instances, the biological sample is incubated in the extraction solution in a humidified cell culture incubator. In some instances, the biological sample is incubated at room temperature. In some instances, the biological sample is incubated in a refrigerator or cooler.

According to some methods of the present disclosure, incubating the biological sample may include agitating the biological sample in the extraction solution for a period of time. For example, a vessel containing the biological sample and the extraction solution may be agitated using vibration, mechanical movement or stirring, or resonant acoustic energy.

In some instances, the biological sample in the extraction solution is agitated using an orbital shaker, a rocker, or a stir plate (with magnetic stirrer in vessel containing biological sample and extraction solution). In some instances, a mechanical impeller agitation system may be used, such as for non-adherent cells. In some instances, the extraction solution is sonicated while the biological sample is contained therein (continuously, for a portion of the incubation period, or intermittently during the incubation period).

In some instances, the agitation system may include a resonant acoustic vibration device that applies resonance acoustic energy to a processing vessel and its contents. The resonant acoustic vibration device introduces acoustic energy into the extraction solution contained by the processing vessel, and the biological sample and extraction solution therein. An exemplary resonant acoustic vibration device is a Resodyn LabRAM ResonantAcoustic® Mixer (Resodyn Acoustic Mixers, Inc., Butte, Montana). In some instances, the resonant acoustic vibration device may be devices such as those described in U.S. Pat. No. 7,866,878 and U.S. Patent Application No. 2015/0146496, which are each incorporated herein in their entireties. Resonant acoustic energy may be applied to a biological sample in solution using the equipment and methods similar to those as described in U.S. Patent Publication Nos. 2017/0035937 and 2018/0280575, which are each incorporated herein by reference in its entirety. Where the processed biological sample comprises viable cells, the parameters of such methods may be selected such that either minimal cell lysis of the cells within the biological sample occurs or such that complete or substantial lysis of the cells within the biological sample occurs.

### 2. Extraction Solution

The biological sample is incubated in an extraction solution. In some instances, the biological sample is incubated in one or more extraction solutions. In some instances, the extraction solution may enhance cell viability and the formed enriched tissue extract by providing nutrients, by providing protective agents, or by removing harmful environmental components. In some instances, the extraction solutions facilitates tissue degradation or formation of the enriched tissue extract. During the incubation, biologically active components are extracted from the biological sample and the resulting extraction solution with the biologically active components there forms an enriched tissue extract. In some instances, the biological active components are secreted from the live cells of the biological sample. In some instances, extraction of the biologically active components are facilitated by the properties of the extraction solution.

In some instances, the extraction solution may be a buffered solution or a cell culture medium. Additional agents may be added to the extraction solution to stabilize the live cells of the biological sample and/or to facilitate extraction of the biologically active components. In some instances, the extraction solution may further include salts, serum, a detergent, a surfactant, an acid, a base, a chelating agent, a protease inhibitor, a phosphatase inhibitor, or a tissue digestive enzyme. In some instances, the extraction solution may comprise one or more antibiotics and/or antimicrobial agents.

In some instances, the extraction solution may comprise a buffered solution. A buffer solution (also referred to as a pH buffer or hydrogen ion buffer) is an aqueous solution consisting of a mixture of a weak acid and its conjugate base, or vice versa. Suitable buffers include, but are not limited to, potassium phosphate, sodium phosphate, phosphate-buffered saline (PBS), sodium citrate, sodium acetate, sodium borate, 2-(N-morpholino) ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), and the like. The pH of the solution is generally in the range of pH 6.4 to 8.3.

In some instances, the extraction solution may comprise a cell culture medium, also referred to as growth medium. There are many cell culture media known in the art. In some instances, the cell culture medium may be selected based on the type of cells present in the biological sample or the intended use of the enriched tissue extract. Exemplary growth medium include, but are not limited to, minimal essential medium (MEM), Dulbecco's Modified Eagle's Medium (DMEM), high glucose DMEM, F-12, and chondrocyte growth medium.

The extraction solution may further include other components. In some instances, such components may help stabilize or maintain the living cells of the biological sample following the stress response. Certain components may be added to facilitate extraction of the biologically active components from the living cells of the biological sample. For example, the extraction solution may further contain salts (e.g., NaCl, KCl, CaCl₂), and salts of Mn²⁺ and Mg²⁺). In some instances, the extraction solution may further include one or both of sodium hydroxide (NaOH) or hydrochloric acid (HCl). In some instances, the extraction may include hydrogen peroxide (H₂O₂). In some instances, the extraction may include a chelating agent such as, for example, ethylenediaminetetraacetic acid (EDTA).

In some instances, the extraction solution may include serum. Serum is a key component for growing and maintaining cells in culture. Containing a mixture of proteins, hormones, minerals and other growth factors, serum is a nutrient boost for cultured cells. Exemplary serum are fetal bovine serum (FBS), fetal calf serum (FCS), and human serum (e.g., pooled human serum, Human Serum—Type AB (Atlanta Biologics). In some instances, the extraction solution may include albumin protein or the like, which may act as a stabilizer or chaperone for the biologically active components extracted into the extraction solution. In some instances, the extraction solution may include platelet lysate (e.g., human platelet lystate). For example, serum and/or platelet lysate can be added to the extraction solution up to 20% (vol/vol).

In some instances the extraction solution may further include a detergent/surfactant. The detergent may include one or more of an ionic, nonionic, amphiphilic, zwitterionic, or chaotropic agent. In some instances, the detergent may be an ionic detergent such as, for example, anionic detergents such as sodium dodecyl sulfate (SDS), deoxycholate, sodium cholate, N-lauroylsarcosine, sarkosyl, and the like, and cationic detergents such as hexdecyltrimethyl ammonium bromide, trimethyl(tetradecyl) ammonium bromide, and the like. In some instances, the detergent may be a nonionic detergent such as, for example, Triton X-100™ ((C₁₄H₂₂O(C₂H+O)ₙ (n=9-10)), n-dodecyl-β-D-maltoside (DDM), digitonin, Tween™-20, or Tween™-80, N,N-bis[3-(D-gluconamido)propyl]cholamide, polyoxyethylene (20) cetyl ether, dimethyldecylphosphine oxide, branched octylphenoxy poly(ethyleneoxy)ethanol, a polyoxyethylene-polyoxypropylene block copolymer, t-octylphenoxypolyethoxyethanol, polyoxyethylene (20) sorbitan monooleate, and the like. In some instances, the detergent may be a zwitterionic detergent such as, for example, CHAPS, amidosulfobetaine, 3-[(3-cholamidopropyl)dimethyl-ammonio]-1-propanesulfonate, and the like. In some instances, the detergent may be urea. In some cases, the surfactant may include a polymer surfactant such as one or more poloxamers.

Buffers, salts, and detergents/surfactants are generally used at concentrations ranging from about 1 μM to about 250 mM. For example, the concentration of a buffer, a salt, or a detergent/surfactant may be about 1 μM, or about 10 μM, or about 100 μM, or about 1 mM, or about 10 mM, or about 25 mM, or about 50 mM, or about 100 mM, or about 250 mM. The concentration may be lower or higher, depending on factors such as the other components of the extraction solution or the intended separation method.

In some instances, the extraction solution may further include a protease inhibitor and/or a phosphatase inhibitor. Such inhibitors block or inactivate endogenous proteolytic and phospholytic enzymes that are released from subcellular compartments during cells lysis and would otherwise degrade proteins of interest and their activation states. Such compounds can target such proteases as, for example, serine proteases, cysteine proteases, serine and cysteine protease, aspartic acid proteases, serine-threonine phosphatases, acidic phosphatases, tyrosine and alkaline phosphatases, aminopeptidases, and metalloproteases. Exemplary inhibitors include, but are not limited to, AEBSF-HCL, aprotinin, bestatin, E-64, leupeptin, pepstatin, PMSF, EDTA, sodium fluoride, sodium orthovanadate, B-glycero-phosphate, and sodium pyrophosphate (available, for example, at ThermoFisher Scientific).

In some instances, the extraction solution may further include a tissue digestive enzyme such as collagenase. Collagenases are enzymes that break the peptide bonds in collagen. They assist in destroying extracellular structures and breaking down tissue structures. The type of collagenase may be selected for use in the extraction solution based on the type of tissue in the biological sample. In some instances, the extraction solution may comprise a ratio of about 325,000 Units collagenase to 1000 cc biological sample. In some instances, the processing solution may comprise a ratio of about 310,000-350,000 Units collagenase to 1000 cc tissue. In some cases, the extraction solution may comprise 325,000 Units collagenase for up to about 1000 cc tissue. In some cases, the extraction solution may comprise 310,000-350,000 Units collagenase for up to about 1000 cc tissue. For example, for up to 1000 cc of tissue, the extraction solution may include 15,000 U; 30,000 U; 35,000 U; 45,000 U; 50,000 U; 55,000 U; 60,000 U, 65,000 U; 70,000 U; 75,000 U; 80,000 U; 85,000 U; 90,000 U; 95,000 U; 100,000 U; 110,000 U; 125,000 U; 130,000 U; 145,000 U; 150,000 U; 160,000 U; 175,000 U; 180,000 U; 190,000 U; 200,00 U; 210,000 U; 225,000 U; 240,000 U; 250,000 U; 260,000 U; 275,000 U, 290,000 U; 307,000 U, or another amount within 10% of any of these amounts. If the amount of biological sample is increased, the amount of collagenase may be increased proportionally.

### D. Separation

As discussed above, the release of biologically active components from the biological sample into the extraction solution during the incubation period results in the formation of an enriched tissue extract (i.e. the extraction solution with the biologically active components therein). For clarity, after the incubation period, the extraction solution is referred to as the enriched tissue extract. In some instances, the methods of the present disclosure may include separating the enriched tissue extract from the biological sample. Any suitable method may be used to separate the enriched tissue extract from the remainder of the biological sample (i.e. tissue debris). For example, separating the enriched tissue extract from the biological sample may include one or both of centrifuging or filtrating the enriched tissue extract and the biological sample.

In one example, the biological sample in the enriched tissue extract therein, may be sieved. The sieve may separate the enriched tissue extract from solid components of the biological sample. Depending on the size of the biologically active components within the enriched tissue extract, the size of the sieve may vary. For example, the sieve may be a 100 μm sieve, 90 μm sieve, 80 μm sieve, 70 μm sieve, 60 μm sieve, 50 μm sieve, 40 μm sieve, 30 μm sieve, 20 μm sieve, or 10 μm sieve. In some instances, a series of sequentially finer sieves may be used to filter the enriched tissue extract to remove solids and particulate matter from 5 micron to 0.22 μm.

In some embodiments, the enriched tissue extract may be separated from the solid components of the biological sample using a centrifuge. For example, the enriched tissue extract maybe centrifuged to pellet the solid component. The enriched tissue extract may be collected as the supernatant. Depending on the nature of the biological sample, the centrifuging cycle may have various operating conditions. For example, in some examples the centrifuging cycle may include centrifuging the biological sample with the enriched tissue extract at 1,500 G, at 2,000 G, at 3,000 G, at 4,000 G, at 5,000 G, at 6,000 G, at 7,000 G, at 8,000 G, at 9,000 G, at 10,000 G, at 11,000 G, at 12,000 G, at 13,000 G, at 14,000 G, at 15,000 G, at 16,000 G, at 17,000 G, at 18,000 G, at 19,000 G, at 20,000 G, 21,000 G, at 22,000 G, at 23,000 G, at 24,000 G, at 25,000 G, or at a G force within 500 G of any of these forces. The time duration of the centrifuging cycle maybe, for example, from 60 minutes to 120 minutes, from 30 minutes to 60 minutes, from 1 minutes to 20 minutes, from 1 minutes to 18 minutes, from 2 minutes to 15 minutes, from 3 minutes to 12 minutes, or from 5 minutes to 10 minutes.

In some instances, separating the enriched tissue extract from the remainder of the biological sample (i.e. tissue debris) may include centrifuging the enriched tissue extract and the biological sample through a sieve. The solid components of the biological sample would remain in the sieve and the enriched tissue extract would flow through the sieve and be collected.

### II. Enriched Tissue Extracts

### A. General Overview of Enriched Tissue Extracts

The enriched tissue extracts include various biologically active components extracted from a biological sample. In some instances, the biological samples has been exposed to a physical stress so as to induce a stress response in the live cells therein prior to being used to prepare the enriched tissue extract. In some instances, the biological sample has been processed to disrupt the integrity of the cellular structure to expose the inner cellular material of at least some of the cells in the biological sample prior to it being used to prepare the enriched tissue extract. In some instances, the biological sample has been processed to kill all or substantially all of the cells therein prior to being used to prepare the enriched tissue extract. The biologically active components may include one or more components produced by the live cells in the biological sample in response to the physical stress or processing of the biological sample. In the context of this disclosure, physical stress including mechanical stress, chemical stress, and/or hypothermic stress is particularly of interest. The enriched tissue extracts may be produced by the methods described in this disclosure. The profile of biologically active components in the enriched tissue extract may vary based on the type of biological sample, whether the biological sample has been exposed to a physical stress and the nature of the physical stress, whether the biological sample included living cells during the extraction step, and the extraction conditions used. For example, living cells may produce enriched tissue extracts having higher concentrations of exosomes, while extracts made from processed biological tissue having few to no living cells may produce enriched tissue extracts having high concentrations of extracellular matrix proteins. Enriched tissue extracts made from biological samples comprising live cells may have a more limited composition of growth factors and cell signaling proteins relative to extracts made from biological samples that do not comprise live cells. The biologically active components in the enriched cell extracts may include one or more of micro-vesicles, exosomes, growth factors, nucleic acids, cytokines, extracellular matrix proteins, or signaling molecules. The signaling molecules may include one or more of amino acids, hormones, cytokines, neurotransmitters, cyclic AMP, or steroids.

The enriched cell extracts have a variety of uses, both clinically and for research purposes. In some instances, the enriched tissue extract may induce cell differentiation. For example, the enriched tissue extract may induce differentiation of mesenchymal stem cells into osteocytes, adipocytes, chondrocytes, and myocytes. Extracts of this disclosure may also promote differentiation of astrocytes and endothelial cells in neural or dermal tissue, respectively, or in culture. The enriched tissue extracts may also influence or promote cellular function. For example, the enriched tissue extracts according to certain embodiments may enhance cellular production of extracellular matrix. The enriched tissue extracts can also be used to reduce or inhibit cellular function. For example, in some embodiments, the enriched tissue extracts may prevent cellular apoptosis. In other embodiments, the enriched tissue extracts can be used to induce cells that are exposed to such extracts to secrete paracrine factors to induce a response in neighboring cells of a different type.

### B. Concentrating the Enriched Tissue Extract

In some instances, the methods of the present disclosure may include concentrating the enriched tissue extract. Any suitable methods of concentrating the enriched tissue extract may be used. For example, the concentrating methods may include one or more of precipitation, cellulose membrane concentration, dialysis, or chromatography. In some embodiments, concentrating the enriched tissue extract may include isolating one or more biologically active component within the extract, such as for example, exosomes, microvesicles, or growth factors.

Suitable methods of precipitation include salting out (using, e.g., sodium chloride or ammonium sulfate) or precipitation by hydrophilic polymers or miscible solvents.

Membrane concentration involves the use of centrifugal filtration or tangential flow filtration with a suitable molecular weight cutoff (MWCO) size for the proteins to either permeate or be retained within the membrane. Exemplary membranes for use in this method are cellulose and polyethersulfone.

Dialysis may be used to concentrate the enriched tissue extract by dialyzing a sample against a suitable dialysate using tubing, cassette, or other enclosure with a suitable MWCO.

Chromatographic methods are well-known in the art and may be used to isolate particular fractions or specific proteins of the enriched tissue extract through use of commercially available columns or automated HPLC methods.

Some embodiments of the present disclosure include methods for producing isolated exosomes or microvesicles (collectively, extracellular vesicles) from the enriched tissue extract. In some embodiments, biologically active components released by live cells in a biological sample (unstressed or subjected to physical stress) can include exosomes and/or microvesicles. Exosomes and microvesicles can influence the response of a tissue to an injury, diseases, or infection (hereinafter referred to as “injured tissue”) when administered to the injured tissue. In particular, exosomes and microvesicles can induce a natural cascading effect of healing within a subject. Exosomes fuse with cell membranes directly and do not require receptors to mediate uptake of pro-healing signals. Accordingly, isolated exosomes and microvesicles produced according to the methods of the present disclosure may produce greater healing effects than conventional therapies for treating injured tissue. They may also be used in cell culture methods to promote cell growth and function.

Any suitable methods of isolating the exosomes and microvesicles may be used. For example, an exosome isolation kit may be used. In some instances, isolating the exosomes may include filter sterilizing the enriched tissue extract through a series of filters. The series of filters may have sequentially reduced pore size. A final filter within the series of filters may have a pore size of 0.4 μm or less, 0.3 μm or less, 0.25 μm or less, 0.22 μm or less, 0.2 μm or less, 0.15 μm or less, or 0.1 μm or less. The pore size of the final filter may be selected such to isolate exosomes and/or microvesicles from the enriched tissue extract. In other cases, the exosomes and/or microvesicles may be isolated from the enriched tissue extract via magnetic cell separation or particle separation. In other cases, exosomes and microvesicles may be isolated from the extract via separation based on fluorescently labeled tags. In other instances, exosomes and microvesicles may be isolated based on hydrophobicity or affinity for vesicle membranes, or lack thereof.

### C. Enriched Tissue Extract Additives

In some instances, the methods of the present disclosure may include adding at least one additive to the enriched tissue extract. In some instances, the additive used in the methods provided herein facilitates enhancement of viability of the enriched tissue extract. For example, an additive to the enriched tissue extract may include at least one of a recombinant growth factor protein, a protease inhibitor, a serum, an antibiotic, or a combination of any thereof.

One of more additives may be added to the enriched tissue extract enhance the biologically active components within the enriched tissue extract. For example, protease inhibitors and phosphatase inhibitors as discussed elsewhere in this disclosure may be added to the extract to preserve the structure and function of the biologically active agents. Serum as discussed above can be added as a protease inhibitor and an additional source of growth factors. Recombinant growth factors may be added to supplement the enriched tissue extract resulting in a composition having greater utility to initiate cellular growth or encourage particular cellular function in vitro or clinically.

In some instances, the additive may be one or more antibiotics. The addition of antibiotics to the enriched tissue extract may facilitate utility of the enriched tissue extract to promote cell growth or cellular function by reducing the incidence of microbial growth.

### D. Cryopreservation of Enriched Tissue Extracts

In some instances, the enriched tissue extract may be frozen or cryopreserved. For example, in some embodiments, the enriched tissue extract may be frozen to a temperature of −20° C. In some cases, the enriched tissue extract may be combined with a cryopreservative solution or cryoprotectant prior to freezing. Cryoprotectants (also referred to as cryoprotective agents, cryoprotectant agents, and cryopreservatives) protect the biological material from the damaging effects of freezing (such as ice crystal formation and increased solute concentration as the water molecules in the biological material freeze).

In some instances, a cryoprotectant may be added directly to the enriched tissue extract. In some instances, the enriched tissue extract may be combined with a cryoprotectant solution that contains a cyroprotectant. Exemplary cryoprotectant agents include, for example, dimethyl sulfoxide (DMSO), methanol, butanediol, propanediol, polyvinylpyrrolidone, glycerol, hydroxyethyl starch, alginate, and glycols, such as, for example, ethylene glycol, polyethylene glycol, propylene glycol, and butylene glycol. In some instances, combinations of more than one cryoprotectant agent may be used. In one example, the cryopreservative solution may include 6 mol ethyene glycol I-1 and 1.8 mol glycerol I-1. In some instances, the cryoprotectant may be a compound that aids in dehydration (e.g., sugars) or formation of a solid state (e.g., polymers, complex carbohydrates). In some instances, the cryopreservation solution may contain 5% to 30% of a cryoprotectant, or combination of cryoprotectants, in a buffer solution such as a buffered solution or cell culture medium. In some instances, the cryopreservative solution may comprise serum or platelet rich plasma, or both, and one or more cryoprotectants. For example, the cryopreservation solution may comprise cell culture medium containing 5-40%, 10-20%, or 10-30% DMSO. In some instances, the cryopreservation solution may contain 5%, 10%, 15%, 20%, 25%, 30%, 35%, or 40% DMSO. In some instances, the cryopreservation solution contains 20% DMSO. The concentration of cryoprotectant in the cryopreservative solution may also vary depending on the type or volume of the enriched tissue extract being cryopreserved.

In some embodiments, the tissue extracts are frozen at a controlled rate, similar to how cells are typically frozen down for cryopreservation. For example, a 1º C per minute cooling rate using a device with controlled rate freezing could be used. Controlled cooling may also be achieved by placing the tissue extract in a container surrounded by alcohol to temper the freezing rate after placing the tissue extract in a freezer (e.g., using a Nalgene® Mr. Frosty™ Freezing Container).

The cryopreservation methods described herein with respect to the enriched tissue extract may also be used for concentrated enriched tissue extract and isolated components thereof, including growth factors, exosomes, and microvesicles.

### III. Methods of Using

The enriched tissue extracts as provided herein may be used for a variety of therapeutics purposes to assist in the healing of tissue wound such as injured or diseased tissue. In particular, the enriched tissue extracts may be administered to human or animal patients to assist in the healing process of tissue. A wound may be a tissue injury, a surgical site in a tissue, or a disease tissue of the subject. In aspects of this disclosure, a method of treating a subject with a wound includes the step of administering the enriched tissue extract or isolated components thereof as described elsewhere in this disclosure, to the wound of a subject. Any suitable means of administration may be used. For example, the enriched tissue extracts may be used to treat a subject with a wound by administering the enriched tissue extract to a wound site or surgical site of a subject. The enriched tissue extracts may be administered by injection, combined with slow release compounds for topical or surgical applications, or may be added to regenerative medical treatments (e.g., combined with live cells) to stimulate stem cells or other primary cells.

In some instances, the enriched tissue extracts may be used with specific types of tissue. Without being tied to any particular theory, in some instances, by forming an enriched tissue extract from a particular type of tissue, the biologically active components within the enriched tissue extract may be specific to the particular type of tissue and, as such, may be well suited for treatment of an injury to that type of tissue in a subject. For example, enriched tissue extract produced from cartilage tissue may better facilitate healing of injured cartilage than enriched tissue extract produce from any other type of tissue because the biologically active component released from chondrocytes within the cartilage biological sample during the stress response may be specific to healing cartilage tissue. The biologically active components may include signaling molecules, growth factors, and hormones that are specific to that tissue type. Thus, utilizing a enriched tissue extract that includes biologically active components that are specific to the type of tissue that is injured may more readily induce the cascade of healing events within the injured tissue.

In exemplary embodiments, enriched tissue extracts produced from tendon tissue may be used as therapeutic compositions to treat tendon tears, ligament tears, muscle tears, bone fractures, wounds to the skin, and to assist in spinal fusion procedures.

In exemplary embodiments, enriched tissue extracts produced from ligament tissue may be used as therapeutic compositions to treat ligament tears, tendon tears, muscle tears, bone fractures, wounds to the skin, and to assist in spinal fusion procedures.

In exemplary embodiments, enriched tissue extracts produced from periosteum tissue may be used as therapeutic compositions to treat bone injury, cartilage injury, osteochondral tissue injury and to assist in spinal fusion procedures.

In exemplary embodiments, enriched tissue extracts produced from dermal tissue may be used as therapeutic compositions to treat dermal scaring, dermatitis, skin grafts, and dermal burns, wound healing, and as a prophylactic for surgical dehiscence.

In exemplary embodiments, enriched tissue extracts produced from fascia tissue may be used as therapeutic compositions to treat muscle tears or volumetric muscle loss.

In exemplary embodiments, enriched tissue extracts produced from nerve tissue may be used as therapeutic compositions to treat brain injury, spinal cord injury, spinal nerve damage, peripheral nerve damage or loss, volumetric muscle loss.

In exemplary embodiments, enriched tissue extracts produced from vascular tissue may be used as therapeutic compositions to treat lung transplants, lobectomies, and lung trauma.

In exemplary embodiments, enriched tissue extracts produced from amniotic tissue may be used as therapeutic compositions to treat skin injuries, ocular surface injuries, spinal injuries, knee injuries, child birth-related injuries, shoulder surgery, spinal surgeries, trauma related cases, cardiovascular procedures, brain/neurological procedures, or burn and wound care.

In exemplary embodiments, enriched tissue extracts produced from adipose tissue may be used as therapeutic compositions to treat ailments requiring a correction of age-, surgery-, and disease-related facial depressions and rhytids (wrinkles) and other conditions that require volume augmentation at other body sites.

In some instances, the enriched tissue extract may be used as part of the preparation or formation of a graft (cells, tissues, synthetic materials) or a stent. For example, the enriched tissue extract may be used to stimulate extracellular matrix formation in a material co-cultured with stem or other cells intended for surgical repair.

The enriched tissue extracts, or isolated components thereof as described elsewhere in this disclosure, can also be used for a variety of laboratory and research applications. In one aspect of this disclosure, provided is a method of in vitro culturing cells, the method including the step of culturing cells in a cell culture medium comprising the enriched tissue extract or isolated components thereof, such as, for example, exosomes, microvesicles, or growth factors. For example, the enriched tissue extracts and isolated components thereof may be used to maintain phenotype and health of primary or immortalized cell populations. In some cases, the enriched tissue extract, or the isolated exosomes and/or microvesicles from the enriched tissue extract, may be used as part of a cell culture medium for in vitro culturing of cells. For example, the enriched tissue extract and isolated components thereof may be used to accelerate growth of cells either as the cell culture medium for as an additive to the cellular medium. The enriched tissue extract and isolated components thereof can be used to culture a variety of cells, such as for example mesenchymal stem cells, tenocytes, myocytes, adipocytes, fibroblasts, osteocytes, or endothelial cells. The cultured cells may include cultured eukaryotic cells which have been cultured in the presence of the enriched tissue extract or isolated components thereof. Research laboratories may benefit from the ability of these enriched tissue extracts to support difficult to maintain primary cell cultures. The enriched tissue extracts and isolated components thereof can also be used for research into tissue and cell function. For example, the extracts can be used to stimulate a cell population to secrete certain factors (e.g., extracellular vesicles, including exosomes and microvesicles) that can be used for research and/or therapeutic use.

In one example, the enriched tissue extract may be used to obtain extracellular vesicles from cultured cells. The extracellular vesicles, such as exosomes and microvesicles, can include proteins expressed by cells and secreted into the extracellular space (i.e. proteins processed by the secretory pathway). Such proteins are part of the cell secretosome and include cytokines, growth factors, extracellular matrix proteins and regulators, and shed receptors. In humans, the secretome includes 13 to 20% of all proteins. The secretome may be particularly influential on initiation the healing cascade within injured tissue. Thus, extracellular vesicles obtained from cultured cells using the enriched tissue extract of the disclosure may be useful for therapeutic purposes. To produce extracellular vesicles, according the methods of this disclosure, cells may be cultured in vitro in a serum-free cell culture medium. The serum-free culture medium may include enriched tissue extract produced according to the present disclosure. After the cells are cultured, a supernatant may be separated from the cells. The supernatant may include the serum-free cell culture medium and extracellular vesicles. Next, the extracellular vesicles may be isolated from the supernatant. Isolation of the extracellular vesicles may be done using any suitable means, such as membrane filtration or pelleting centrifugation.

### EXAMPLES

### Example 1. Preparation of Enriched Tendon Extract from Tendon Tissue

This example describes methods used to prepare enriched tendon extract from tendon tissue. All extracts were tested after cryopreservation as described in Examples 2-4 below.

This study was performed using consented tendon tissue obtained from deceased human donors. The tendon tissue was removed from a donor and transported aseptically on ice. The tendon tissue was processed prior to cellular death (e.g., 24-72 hours after removal from donor). Contaminating tissue, such as bone an fascia, was removed manually and then the tendon tissue was cleansed using a propriety Allotrue™ method that which utilizes various detergents, ethanol, and antibiotics and washes under different flow and pressure. After cleaning, the tendon tissue was cut into 1-3 cm segments. The segmented tendon tissue was cryofractured by placing the tissue segments into a liquid nitrogen bath for less than one minute (e.g., 15 to 20 seconds) and then grinding it using a mechanical grinder (blender). Due to the frozen state of the segmented tendon tissue, the tendon tissue was brittle and fractured cleanly into small-diameter fibers or varying lengths. Due to the freezing of the segmented tendon tissue, all cells within the tendon tissue were dead prior to extracting the biologically active factors.

The ground tendon tissue was then mixed with serum free cell culture medium. The combination of ground tendon tissue and saline solution incubated for 60 minutes at 37° C. with continuous stirring to allow the biologically active components to be extracted from the live cells of the tendon tissue.

After the incubation, a liquid portion was separated from solid fibers in the mixture by centrifugation through a 40 μm sieve. Next, the liquid portion was centrifuged for 15 minutes at 3,000 g to separate solids from a supernatant. The supernatant is the enriched tissue extract.

### Example 2. Analysis of Enriched Tendon Extract on Tenocytes in Culture

The purpose of this study was to assess the impact of enriched tendon extract on the growth of tenocytes in culture. Standard procedures for growing tenocytes use commercially available serum free medium and have lengthy processing times (e.g., 14-21 days). The hypothesis evaluated in this study was whether the use of enriched tendon extract may increase the rate of in vitro tenocyte growth rate. Tenocyte growth rate was assessed by the number of cells per well grown after 7 days.

Enriched Tissue Extract Preparation. This study was performed using consented tendon tissue obtained from deceased human donors. The tendon tissue was cleansed by the propriety Allotrue™ process described in Example 1 and then cut into 1 cm segments. An enriched tissue extract was prepared from the tendon tissue as described in Example 1.

Tenocyte Cultures. Tenocytes were obtained using explant cultures at AlloSource using minced fresh tendon tissue from a deceased human donor. After serial passaging of tenocytes, 100,000 cells were seeded per well in 6 well plates. Each well included a total volume of 5 ml of commercially available serum free medium (DMEM/F-120) supplemented as noted below. The conditions assessed were: (1) unsupplemented medium, which was used to establish a base growth rate for tenocytes, (2) medium supplemented with 5% by volume of enriched tendon extract, (3) medium supplemented with 10% by volume of fetal bovine serum (Sigma), and (4) medium supplemented with 10% by volume fetal bovine serum and 5% by volume of enriched tendon extract. Three wells were prepared per condition. The cultures were grown under standard cell culture conditions (37° C. in an atmosphere containing 5% by volume CO₂ and ambient O₂ levels) for 7 days. The medium was changed on day 4 of the 7-day period.

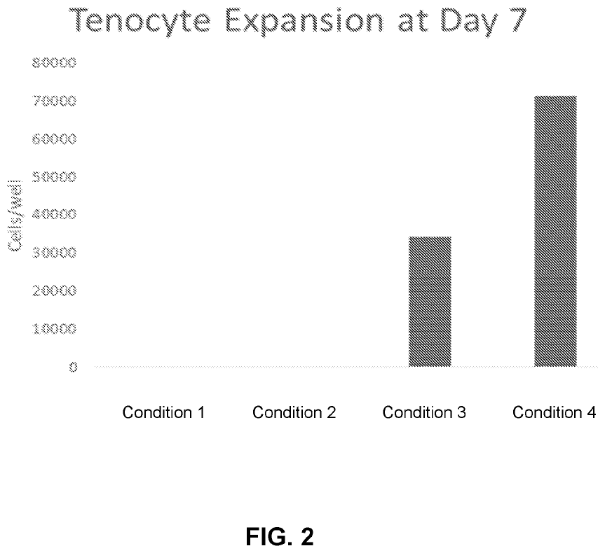

Results. After 7 days of incubation, each of the culture samples were evaluated to assess cell growth. Cells were trypsinized and the cell numbers were counted manually counting per well/condition (representative) with a hemacytometer. Images were also taken and used for qualitative assessment. The data is shown in FIG. 2. Serum free medium alone (condition 1) did not support tenocyte growth. The serum free medium with 5% enriched tendon extract (condition 2) also did not support tenocyte growth. Conditions 3 and 4 did supported tenocyte growth. Medium with 10% by volume of fetal bovine serum (condition 3) supported approximately 1.2 doubling of cells in 7 days. This is consistent with literature values. Per Yao et al., “Phenotypic drift in human tenocyte culture,” TISSUE ENGINEERING, Vol. 12, No. 7, pp. 1843-1849 (2006), cell doubling in DMEM with 10% by volume fetal bovine serum is expected within 126 hours. Medium with 10% fetal bovine serum and 5% enriched tendon extract (condition 4) exhibited 2.3 doubling of the cells in 7 days. This exceeds the predicted doubling rate in literature.

### Example 3. Analysis of Biological Activity of Enriched Tendon Extract on Bone Marrow Derived Mesenchymal Stem Cells

The purpose of this study was to assess the biological activity of enriched tendon extract on bone marrow derived mesenchymal stem cells (MSCs) (harvested from cancellous bone in the femur of a deceased human donor. The hypothesis evaluated in this study was whether the use of enriched tendon extract may increase biological activity of MSCs. Standard protocols were followed to obtain the mesenchymal stem cells from bone marrow. The biological activity was evaluated by visually assessing the amount of extracellular matrix formed by the MSCs under various conditions. An increase in extracellular matrix amount corresponds to increased biological activity. Increases in biological activity may represent increased healing rate for in vivo tissue.

Enriched Tissue Extract Preparation. The enriched tissue extract was prepared in accordance to the methods described in Example 2.

MSC Cultures. A 6 well plate was seeded with 100,000 bone marrow derived MSCs in each well. To each well, 5 ml of serum free MSC media (e.g., Human Mesenchymal-XF Expansion Medium, EMD Millipore) was added along with a supplemental amount of enriched tendon extract. The conditions assessed were: (1) unsupplemented medium, which was used to establish a base activity rate, (2) medium supplemented with 5% by volume enriched tendon extract, (3) medium supplemented with 10% by volume enriched tendon extract, and (4) medium supplemented with 20% by volume enriched tendon extract. The cultures were grown under standard cell culture conditions as noted in Example 2 for 4 days. Three wells were prepared per condition.

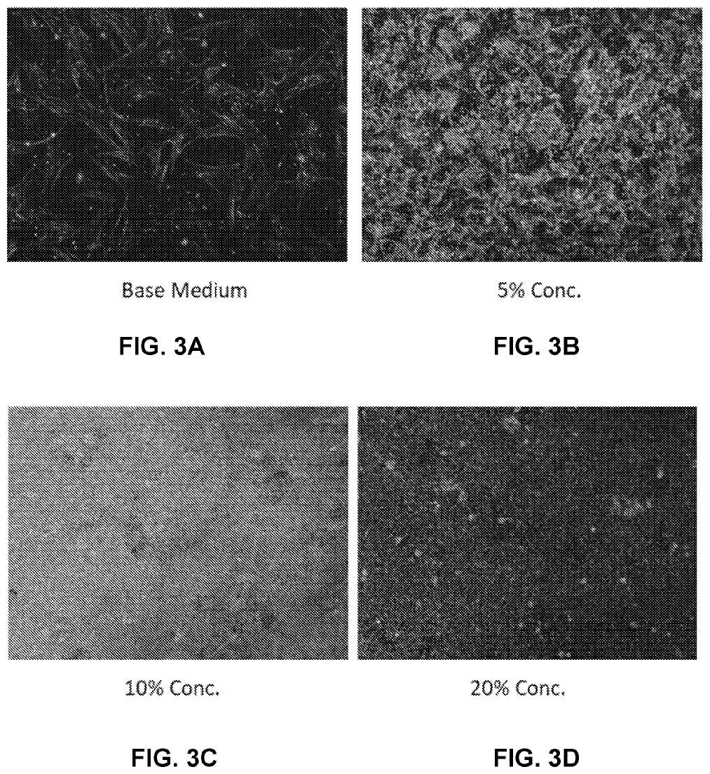

Results. After 4 days of incubation, each of the culture samples were evaluated by bright field microscopy (image contrasts were enhanced by manipulation using ImageJ software). Representative images are shown in FIGS. 3A-3D.

As shown by FIG. 3A, cells cultured in standard base medium exhibited minimal extracellular matrix formation. FIGS. 3B-3D depict images of extracellular growth for cells cultured using medium containing various amounts of enriched tendon extract. As the images show, there was significant biological activity when the enriched tendon extract is added to the cell culture medium. Specifically, the cultured cells produced visible amounts of extracellular matrix in the presence of the enriched tendon extract. As the concentration of enriched tissue extract increased in the cell culture medium, the visible amount of extracellular matrix produced by the MSCs also increased.

### Example 4. Analysis of Attachment of Bone Marrow Derived Mesenchymal Stem Cells to Tendon Tissue Treated With Enriched Tendon Extract

The purpose of this study was to assess whether cultured mesenchymal stem cells (MSC) would attach to tendon tissue soaked in an enriched tendon extract to a greater or lesser extent than they would attach to an untreated tendon tissue (soaked in phosphate buffered saline).

Tendon Tissue. This study was performed using semitendinosus tendons obtained from a single deceased human donor. The tendon tissue was cleansed and partially decellularized using the propriety Allotrue™ method described in Example 1. The tissue was then stored at −20° C. and subsequently thawed for experimentation. The tissue was then cut into 1-2 cm long segments.

Enriched Tissue Extract Preparation. The enriched tissue extract was prepared in accordance to the methods described in Example 2.

Culture Assay. One tissue segment was distributed into each well of a 6 well plate. Segments were soaked for 16 hours at 4ºC in one of the following soak solutions: (1) phosphate buffered saline (PBS); (2) PBS supplemented with 10% by volume tendon extract; or (3) PBS supplemented with 50% by volume tendon extract. Tendon segments were then rinsed three times with PBS.

Subcultured human adipose-derived MSCs (originally purchased from (RoosterBio Inc.) were used in this experiment. The cells were trypsinized, stained with CellTracker™ Red Dye (ThermoFisher) (a red fluorescent dye that stains living cells), and then resuspended in DMEM/F12 growth medium for seeding.

The soak solution was aspirated from the tissue pieces and then samples from each soak condition were treated as one of two “seeding groups”—either covered in plain DMEM/F12 medium (no cells) or in DMEM/F12 medium containing approximately 500,000 MSCs per tissue segment. The tissue segments were incubated at 37° C. under standard cell culture conditions as noted in Example 2. For each seeding group, one tendon segment was soaked for 1 hour and the other tissue segment was soaked for 2 hours, both soaked under mild agitation. This provided the MSCs time to attach to the tendon segments. After incubation, the tendon segments were rinsed three times in PBS so that any non-attached cells were removed and only attached cells remained on the tendon segment. Following the rinsing, complete growth medium was added to cover the tendon segments so that the attached cells maintained viability. To assess attachment of the MSC to the tissue, tendon segments were imaged using a confocal microscope to detect red fluorescence.

Results. The tendon segments that had been soaked in tendon extract (either concentration) showed a higher fluorescent signal than segments soaked in PBS, indicating qualitatively a higher number of attached MSCs to the extract-soaked substrates than the controls (data not shown).

### Example 5. Analysis of Biological Activity of Enriched Bone Marrow Extract on Bone Marrow Derived Mesenchymal Stem Cells

The purpose of this study was to assess the biological activity of enriched bone marrow extract on bone marrow derived mesenchymal stem cells (MSCs). The hypothesis evaluated in this study was whether the use of enriched bone marrow extract may increase biological activity of MSCs derived from bone marrow. The bone marrow MSCs were obtained according to the methods of Example 3. The biological activity was evaluated by visually assessing the amount of extracellular matrix formed by the MSCs. An increase in extracellular matrix amount corresponds to increased biological activity. Increases in biological activity may represent increased healing rate for in vivo tissue.

Enriched Tissue Extract Preparation. The enriched bone marrow extract was prepared using cancellous bone obtained from deceased human donors. The cancellous bone was ground without freezing, meaning that at least a portion of the cells within the cancellous bone were alive during extraction. The ground bone was then combined with an equal amount (weight:volume) of cell culture medium (DME/F-12, 10% FBS, 1% Antibiotic-Antimycotic (100×, containing penicillin, streptomycin, and amphotericin B) (Thermo Fisher)) (extraction solution) and incubated for 1 hour with agitation in a rotational shaker at 50 RPM at a temperature of 37° C. After incubation, the mixture was filtered using a 22 μm mesh sieve to remove cellular and solid components from and sterilize the enriched bone extract.

MSC Cultures. A 6 well plate was seeded with 100,000 bone marrow derived MSCs in each well. To each well, 5 ml of serum free MSC media (e.g., Human Mesenchymal-XF Expansion Medium, EMD Millipore) was added along with a supplemental amount of enriched bone extract. The conditions assessed were: (1) medium supplemented with 10% by volume enriched bone extract, (2) medium supplemented with 20% by volume enriched bone extract, and (3) medium supplemented with 40% by volume enriched bone extract. As a negative control, MSC were fixed using formalin (i.e. killed) and incubated in medium supplemented with 40% by volume enriched bone extract. The cultures were grown under standard cell culture conditions as noted in Example 2 for 24 hours.

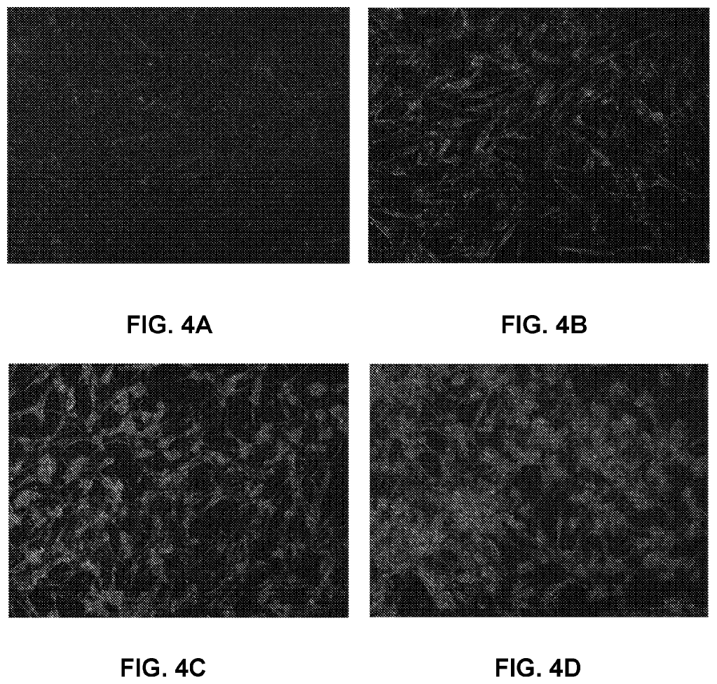

Results. After 24 hours of incubation, each of the culture samples were evaluated by bright field microscopy (image contrasts were enhanced by manipulation using ImageJ software). The images as shown in FIGS. 4A-4D illustrate the results of this experiment.

As shown by FIG. 4A, the negative control exhibited minimal extracellular matrix formation. FIGS. 4B-4D depict images of extracellular growth for cells cultured using medium containing various amounts of enriched bone extract. As the images show, there was significant biological activity when the enriched bone extract is added to the cell culture medium. Specifically, the cultured cells produced visible amounts of extracellular matrix in the presence of the enriched bone extract. As the concentration of enriched bone extract increased in the cell culture medium, the visible amount of extracellular matrix produced by the MSCs also increased.

### Example 6. Analysis of Biological Activity of the Exosome Fraction of Enriched Bone Marrow Extract on Bone Marrow Derived Mesenchymal Stem Cells

The purpose of this study was to assess the biological activity of the exosome fraction of enriched bone marrow extract on bone marrow derived mesenchymal stem cells (MSCs). The hypothesis evaluated in this study was whether the use of the exosome fraction of enriched bone marrow extract may increase biological activity of MSCs derived from bone marrow. The bone marrow MSCs were obtained according to the methods of Example 3. The biological activity was evaluated by visually assessing the amount of extracellular matrix formed by the MSCs. Visual comparison focused on the amount of “debris” or material that formed above the cells, thus blocking the view of the underlying cells. An increase in extracellular matrix amount corresponds to increased biological activity. Increases in biological activity may represent increased healing rate for in vivo tissue.

Enriched Tissue Extract Preparation. Using the enriched bone marrow extract produced according to the methods of Example 5, an exosome fraction was isolated using an exosome isolation kit (ExoQuick-TC™, System Biosciences). The exosome fraction was cryopreserved in a trehalose-based cryopreservation medium (−80° C.). A second portion of the enriched tissue extract was frozen (−80° C.) without being combined with a cryopreservation medium.

MSC Cultures. A 6 well plate was seeded with 100,000 bone marrow derived MSCs in each well. To each well, 5 ml of serum free MSC media was added along with a supplemental amount of the exosome fraction. The conditions assessed were: (1) medium supplemented with 10% by volume exosome fraction, (2) medium supplemented with 20% by volume exosome fraction, and (3) medium supplemented with 40% by volume exosome fraction. As a negative control, MSC were fixed using formalin (i.e. killed) and incubated in medium supplemented with 40% by volume exosome fraction. The cultures were grown at standard cell culture conditions as noted in Example 2 for 24 hours.

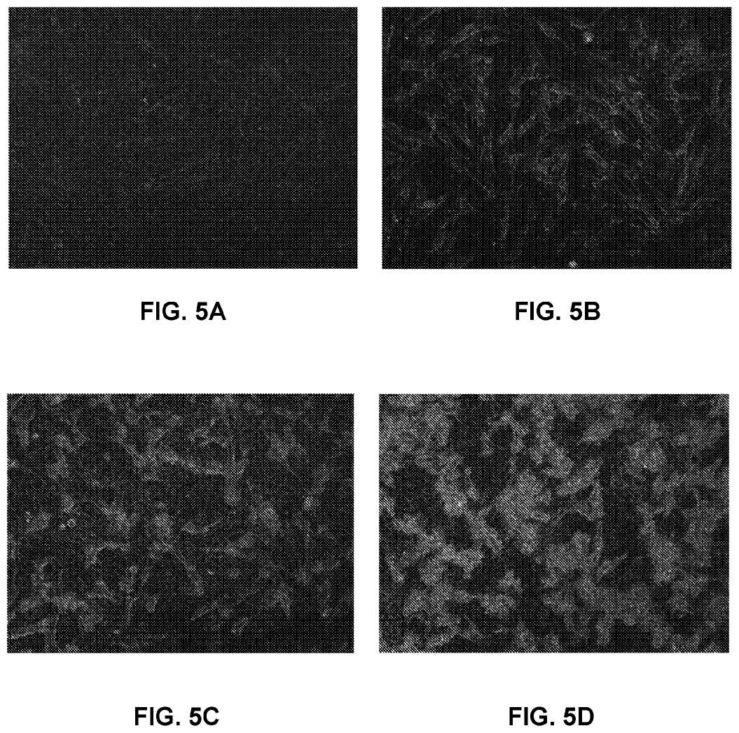

Results. After 24 hours of incubation, each of the culture samples were evaluated by bright field microscopy (image contrasts were enhanced by manipulation using ImageJ software). The images as shown in FIGS. 5A-5D illustrate the results of visual inspection of the samples

As shown by FIG. 5A, negative control exhibited minimal extracellular matrix formation. FIGS. 5B-5D depict images of extracellular growth for cells cultured using medium containing various amounts of the exosome fraction from enriched bone extract. As the images show, there was significant biological activity when the exosome fraction is added to the cell culture medium. Specifically, the cultured cells produced visible amounts of extracellular matrix in the presence of the exosome fraction from the enriched bone extract. As the concentration of exosome fraction increased in the cell culture medium, the visible amount of extracellular matrix produced by the MSCs also increased. The results show bioactivity equivalency between the enriched tissue extract itself and the exosome fraction isolated from the enriched tissue extract.

This data shows that the presence of enriched tissue extract and exosome fraction of the enriched tissue extract increases biological activity. That is, the enriched tissue extract stimulates biological activity within cells which corresponds to healing functions of cells. Thus, increased biological activity is indicative of increased cellular healing. These tests show that the enriched tissue extract can also be used to maintain cell function, stimulate cell cultures to expand at a faster rate, and stimulate cell cultures to produce extracellular matrix.

All patents, patent publications, patent applications, journal articles, books, technical references, and the like discussed in the instant disclosure are incorporated herein by reference in their entirety for all purposes.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“About” is used to provide flexibility to a numerical range endpoint by providing that a given value may be “slightly above” or “slightly below” the endpoint without affecting the desired result. The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

Different arrangements of the components depicted in the drawings or described above, as well as components and steps not shown or described are possible. Similarly, some features and sub-combinations are useful and may be employed without reference to other features and sub-combinations. Embodiments of the disclosure have been described for illustrative and not restrictive purposes, and alternative embodiments will become apparent to readers of this patent. Accordingly, the present disclosure is not limited to the embodiments described above or depicted in the drawings, and various embodiments and modifications can be made without departing from the scope of the claims below.

What is claimed is:

1. A method of producing an enriched tissue extract, the method comprising:
(a) providing a biological sample;
(b) incubating the biological sample in an extraction solution for a period of time sufficient for biologically active components to be extracted from the biological sample thereby forming an enriched tissue extract; and
(c) separating the enriched tissue extract from the processed biological sample.

2. The method of claim 1, wherein the biological sample comprises live cells.

3. The method of claim 2, wherein a step of administering a physical stress to the biological sample occurs prior to incubation in step (b), wherein the physical stress comprises one or more of a mechanical stress, a chemical stress, or a temperature stress.

4. The method of claim 3, wherein the step of administering a physical stress to the biological sample induces a stress response in the live cells.

5. The method of claim 3, wherein the step of administering a physical stress to the biological sample results in minimal cell death.

6. The method of claim 3, wherein the step of administering a physical stress to the biological sample kills all or substantially all of the cells in the biological sample.

7. The method of any one of claims 1-6, wherein the biological sample comprises tissue or isolated cells from one or more of cancellous bone, cortical bone, cortical and cancellous bone, periosteum, ligament, tendon, muscle, placenta, amnion, or umbilical tissue.

8. The method of claim 7, wherein the biological sample comprises a single type of tissue and is substantially free of other types of tissue.

9. The method of claim 7, wherein the biological sample comprises a portion of tissue or a plurality of tissue pieces.

10. The method of claim 7, wherein the biological sample is isolated cells.

11. The method of claim 10, wherein the isolated cells are primary cells.

12. The method of claim 10, wherein the isolated cells are immortalized cells.

13. The method of any one of claims 10-12, wherein the isolated cells are mesenchymal stem cells.

14. The method of claim 13, wherein the isolated cells are immortalized mesenchymal stem cells that are genetically modified to express at least one recombinant growth factor that is not normally expressed by mesenchymal stem cells or to overexpress a growth factor that is normally expressed by mesenchymal stem cells.

15. The method of any one of claims 1-14, wherein the biological sample is from a deceased donor subject.

16. The method of any one of claims 1-14, wherein the biological sample is from a living donor subject.

17. The method of any one of claims 1-16, wherein the biological sample is from a human donor subject.

18. The method of any one of claims 3-17, wherein administering a physical stress to the biological sample comprises at least one of exposing the biological sample to sinusoidal tension, exposing the biological sample to compression or pressure, homogenizing the biological sample, exposing the biological sample to mechanical impacts, or exposing the biological sample to resonant acoustic energy.

19. The method of any one of claims 3-17, wherein administering a physical stress to the biological sample comprises exposing the biological sample to hypothermic temperatures, hyperthermic temperatures, acidic conditions, osmotic stress, non-physiological pH, or non-physiological oxygen levels.

20. The method of any one of claims 3-17, wherein administering a physical stress to the biological sample comprises homogenizing, freezing and thawing the biological sample, cryofracturing the biological sample, or heating the biological sample above 45° C., and wherein administering the physical stress results in death of all or substantially all of the cells present in the biological sample.

21. The method of any one of claims 3-17, wherein the biological sample is bone tissue, and wherein administering a physical stress to the biological sample comprises demineralizing the bone tissue.

22. The method of any one of claims 2-17, wherein the biological sample is exposed to a growth factor prior to incubation in step (b).

23. The method of any one of claims 1-21, wherein the extraction solution comprises a buffered solution or a cell culture medium.

24. The method of any one of claims 1-22, wherein the extraction solution comprises a salt, a serum, a detergent, or a protease inhibitor.

25. The method of any one of claims 1-23, wherein the incubating step comprises soaking the biological sample in an extraction solution for a period of 5 minutes to 24 hours at 2° C.-42° C.

26. The method of any one of claims 1-24, wherein the incubating step comprises agitating the sample in an extraction solution.

27. The method of any one of claims 1-25, wherein the separating step comprises separating the enriched tissue extract from the biological sample using at least one of centrifugation or filtration.

28. The method of any one of claims 1-42, wherein the enriched tissue extract comprises one or more of micro-vesicles, exosomes, growth factor proteins, nucleic acids, extracellular matrix proteins, or signaling molecules.

29. The method of claim 27, wherein the signaling molecules comprises one or more of amino acids, hormones, neurotransmitters, cyclic AMP, or steroids.

30. The method of any one of claims 1-28, further comprising adding to the enriched tissue extract at least one of a recombinant growth factor protein, a protease inhibitor, or a serum.

31. The method of any one of claims 1-29, further comprising concentrating the enriched tissue extract by one or more of precipitation, cellulose membrane concentration, dialysis, or chromatography.

32. The method of any one of claims 1-30, further comprising freezing the enriched tissue extract at −20° C.

33. The method of claim 31, wherein the enriched tissue extract is combined with a cryopreservation medium prior to freezing.

34. An enriched tissue extract produced by the method of any one of claims 1-32.

35. A method of producing isolated extracellular vesicles the method comprising:
(a) producing the enriched tissue extract of any one of claims 1-33; and
(b) isolating extracellular vesicles from the enriched tissue extract.

36. The method of claim 34, wherein the isolating step comprises one or more of filtration, magnetic cell separation, or particle separation.

37. A method of treating a subject with a wound, the method comprising administering to the wound of the subject the enriched tissue extract of claim 33.

38. A method of treating a subject with a wound, the method comprising administering to the wound of the subject the isolated extracellular vesicles produced by the method of claim 34 or 35.

39. A method of in vitro culturing cells, the method comprising culturing cells in a cell culture medium comprising the enriched tissue extract of claim 33 or the isolated extracellular vesicles produced by the method of claim 34 or 35.

40. The method of claim 39, wherein the cells that are cultured comprise one or more of mesenchymal stem cells, tenocytes, fibroblasts, or osteocytes.

41. A method of producing extracellular vesicles, the method comprising:
(a) in vitro culturing cells in a serum-free cell culture medium comprising the enriched tissue extract of claim 33 or the extracellular vesicles produced by the method of claim 34 or 35, and
(b) separating a supernatant comprising the serum-free cell culture medium and extracellular vesicles from the cells; and
(c) isolating the extracellular vesicles from the supernatant by membrane filtration or pelleting by centrifugation.

42. A product comprising cultured eukaryotic cells that have been cultured in the presence of the enriched tissue extract of claim 33 or the extracellular vesicles produced by the method of claim 34 or 35.
